(19) United States
(12) Patent Application Publication
(10) Pub. No.: US 2026/0126385 A1
(43) Pub. Date: May 7, 2026

(54) METHODS AND COMPOSITIONS FOR ENHANCED AND SELECTIVE PHOTOBLEACHING

(71) Applicant: BRUKER NANO, INC., GOLETA, CA (US)

(72) Inventors: Arne Christians, Goleta, CA (US); Thore Boettke, Goleta, CA (US); Jannik Boog, Goleta, CA (US); Oliver Braubach, Goleta, CA (US)

(21) Appl. No.: 19/424,856

(22) Filed: Dec. 18, 2025

Related U.S. Application Data
(63) Continuation of application No. PCT/US2025/038528, filed on Jul. 21, 2025.
(60) Provisional application No. 63/674,750, filed on Jul. 23, 2024.

(51) Int. Cl. G01N 21/64; G01N 1/44; G01N 33/58

(52) U.S. Cl. CPC G01N 21/6428; G01N 1/44; G01N 33/582; G01N 2021/6441

(57) ABSTRACT

Disclosed herein are compositions, systems, kits, and methods for photobleaching, and selective photobleaching, a biological sample.

### CROSS-REFERENCE TO RELATED APPLICATIONS

This application is a continuation of International Application PCT/US2025/038528, filed Jul. 21, 2025, which claims the benefit of U.S. Application 63/674,750, filed Jul. 23, 2024. The contents of the aforementioned applications are incorporated herein by reference in their entireties.

### BACKGROUND

Various methods may be used in biology and in medicine to observe different targets in a biological sample. For example, analysis of proteins and nucleic acids in various tissue or cell preparations may be performed using the techniques of histochemistry, immunohistochemistry (IHC), immunofluorescence (IF), and numerous nucleic acid hybridization, amplification, and visualization techniques. Analysis of proteins and nucleic acids in biological samples may also be performed using solid-state assays, for example, using the techniques of western and northern blots.

Many of the current techniques may detect only a few targets at one time (such as immunohistochemistry (IHC) or fluorescence-based western blots where the number of targets detectable is limited by the fluorescence-based detection system) in a single sample. Further analysis of targets may require use of additional biological samples from the source, limiting the ability to determine relative characteristics of the targets such as the presence, absence, concentration, and/or the spatial distribution of multiple biological targets in the biological sample. Moreover, in certain instances, a limited amount of an individual sample may be available for analysis, or the individual sample may require further analysis. Thus, methods, agents, and devices capable of iteratively analyzing an individual sample are needed.

### SUMMARY

Disclosed herein are methods and compositions useful in the detection, visualization, and/or characterization of target molecules in a biological sample. The methods and compositions include PMS, PDS, a salt thereof, or any combination thereof, wherein PMS and PDS are represented by the following formulas:

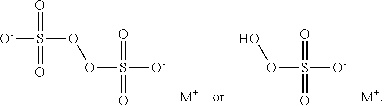

In some embodiments, a method for detection of one or more targets in a biological sample is provided, the method comprising: (a) contacting a biological sample with a detectable label and linking the detectable label to the one or more targets, (b) detecting a signal from the detectable label, (c) after detection at step (b), contacting the sample of (b) with PMS, PDS, a salt thereof, or any combination thereof, wherein PMS and PDS are represented by the following formulas:

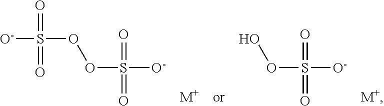

(d) irradiating the sample of (c), wherein irradiating comprises exposing the sample to light, optionally, wherein the light is a selected wavelength range, wherein the selected wavelength range is within the wavelength range required to detect the detectable label; and (e) optionally, repeating steps (a)-(d).

In some embodiments, a method for the sequential detection of multiple targets in a biological sample is provided, the method comprising: (a) contacting a first target probe to the sample, wherein the first target probe is specific for a first target; (b) hybridizing or binding the first target probe to the first target in the sample; (c) detecting a first fluorescent detectable label, wherein the first fluorescent detectable label is linked to the first target probe; (d) after detection at step (c), contacting the sample of (c) with PMS, PDS, a salt thereof, or any combination thereof, wherein PMS and PDS are represented by the following formulas:

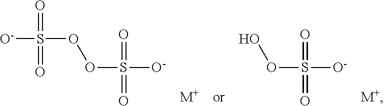

(e) irradiating the sample of (d), wherein irradiating comprises exposing the sample to light, optionally wherein the light is a selected wavelength range of light, wherein the selected wavelength range is within the wavelength range required to detect the first fluorescent detectable label; (f) repeating steps (a)-(e), comprising an Nth target probe and an Nth fluorescent detectable label different from the first probe and first fluorescent detectable label, optionally, wherein the selected wavelength range of light is within the wavelength range required to detect the Nth fluorescent detectable label.

In some embodiments, a method for detecting a plurality of targets in a biological sample is provided, the method comprising: (a) contacting a plurality of target probes to the sample comprising N subsets of target probes, wherein each probe of the subsets of target probes is specific for different target; (b) hybridizing or binding the target probes to the plurality of targets in the sample; (c) detecting a first fluorescent detectable label, wherein the first fluorescent detectable label is linked to a first subset of the plurality of target probes; (d) after detection at step (c), contacting the sample of (c) with PMS, PDS, a salt thereof, or any combination thereof, wherein PMS and PDS are represented by the following formulas:

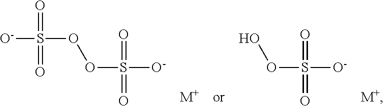

(e) irradiating the sample of (d), wherein irradiating comprises exposing the sample to a light, optionally wherein the light is a selected wavelength range of light, wherein the selected wavelength range is within the wavelength range required to detect the first fluorescent detectable label; and (f) repeating steps (c)-(e), comprising an Nth fluorescent detectable label linked to an Nth probe, wherein the Nth fluorescent detectable label and the Nth subset of target probes are different from the first subset of target probe and the first fluorescent detectable label, optionally wherein the selected wavelength range of light is within the wavelength range required to detect the Nth fluorescent detectable label.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A-1B. Iterative multiplexing strategies to overcome spectral overlap in IF applications. (A) Fluorescent reporter probes allow for selective imaging of ≤5 target probes in standard IF applications. Detection of more targets is problematic due to overlap in fluorescent signals from individual probes (see overlap in emission spectra). (B) Iterative imaging approaches permit higher degrees of multiplexing. This occurs via the deployment of iterative reporter probe binding and signal removal steps. Signal removal approaches include irradiation, antibody elution and antibody barcoding.

FIG. 2. Activation of peroxymonosulfate (PMS) and peroxydisulfate (PDS) by excited dyes. After excitation of the dye by visible light (Dye*), the excited dye activates PMS or PDS by an electron transfer process. PMS and PDS decompose to form sulfate radicals, which in turn react with the dye molecule; this reaction leads to the oxidization and decolorization of the dye. In this system, the dye has a bifunctional role as both a photosensitizer and a substrate for radicals originating from activated PMS and PDS.

FIG. 3 Schematic overview of an iterative multiplexing system with chemically enhanced photobleaching. In Step 1, biological samples are prepared and mounted on carrier slides or chambers that are fitted onto a microscope stage. Images of the endogenous tissue fluorescence (background signal) are recorded using multiple detection channels. Step 2, tissue labeling with target and reporter probes directed at endogenously expressed biomarkers. Step 3, optical detection and recording of reporter probe signals in one or more acquisition channels. Step 4, the reporter probe signal removal through enhanced photobleaching. Enhanced photobleaching includes a signal irradiation process in which the decolorization of a reporter probe signal is accelerated by the presence of a photosensitizing molecule, depicted here as PMS or PDS. Step 5, acquisition of another background signal. Steps 2-5 may be repeated n times. Photobleaching can be performed by irradiation with white light to photobleach all reporter probe signals simultaneously, or with one or more selected wavelengths of light, for selective photobleaching.

FIG. 4A-4B: Selective photobleaching of fluorophores with different wavelength LEDs or white light in vitro. Dyes were dissolved at a concentration of 5 μM with 5 g/l sodium peroxydisulfate (SPS, 21 mM) in PBS. (A) Irradiation was performed with selectively tuned LEDs (refer to legend), set to an output power of 2.52 mW/cm² for up to 20 minutes. Fluorescence of the dye solution was measured after 0, 1, 2, 5, 10, 15 and 20 minutes on an Agilent Biotek Synergy Neo2 fluorescence plate reader. Selective photobleaching was most effective with the LED that has an emission spectrum within the approximate excitation wavelength of the respective fluorophore. LEDs at other wavelengths may also be used to irradiate the fluorophores, but they are less effective. (B) For white light exposures, irradiation was performed with a broad-spectrum white light LED (430-750 nm) set to an output power of 1600 mW/cm². This result demonstrates that the combination of SPS+dye excitation at its preferred wavelength is necessary and effective to produce selective decolorization of organic fluorescent dyes. This result is distinguished from Gao1, where white light is used to decolorize dyes in a non-selective manner. All results are shown as the mean±SD of n=3.

FIG. 5A-5B Selective bleaching of AF594 demonstrates wavelength-dependent SPS photobleaching in vitro. (A) A mixture of AF488 and AF594 with 5 g/l sodium peroxydisulfate (21 mM) in PBS was irradiated with a 590 nm LED (2.52 mW/cm²) for up to 20 minutes. While AF594 showed a fluorescence signal reduction by ˜90% after 20 min of light exposure, AF488 showed no signal reduction. (B) Exposure of the same mixture of dyes and SPS to white light (LED 430-750 nm, 1600 mW/cm²) resulted in signal reduction of both AF488 and AF594. These results demonstrate selective and highly localized reactivity of certain fluorophores under wavelength-specific irradiation in a buffered SPS solution. Result (A) is strong evidence that activation of SPS and subsequent decolorization requires excitation of the dye molecules by exposure to their specific excitation wavelength in the presence of SPS. It also indicates that the decolorization reaction is locally restricted to the excited dye molecules and does not affect non-excited molecules of another dye with different spectral properties in the same solution. The result in (A) is clearly distinguished from Gao1, where only white light is used to decolorize dyes, in a non-selective manner. All results are shown as the mean±SD of n=3.

FIG. 6. Influence of the SPS concentration on bleaching efficiency in vitro. Dyes were added at a concentration of 5 μM to PBS containing varying sodium peroxydisulfate concentrations ranging from 0-10 g/l. Irradiation for purposes of photobleaching was performed for each dye with the optimal LED set to full power (470 nm: 13.17 mW/cm²; 525 nm: 8.82 mW/cm²; 590 nm: 2.52 mW/cm²; 630 nm: 7.2 mW/cm²) over a course of up to 20 minutes. The fluorescence intensity was measured at t=0 minutes, prior to irradiation, and the normalized fluorescence intensity value was set to 100%. Each tested fluorophore showed good photostability during photobleaching without SPS (blue line), while increasingly accelerated photobleaching was triggered by the addition of SPS in a concentration-dependent manner. These data demonstrate that SPS is responsible for the observed bleaching during selective fluorophore illumination/irradiation. The higher the concentration, the better the decolorization when using SPS. All results are shown as the mean±SD of n=3.

FIG. 7. Influence of bleaching light intensity on bleaching efficiency in vitro. Dyes were used at a concentration of 5 μM with 5 g/l sodium peroxydisulfate (21 mM) in PBS. Irradiation for purposes of photobleaching was performed for each dye with the optimal LED set to 2.52 mW/cm² or full power (470 nm: 13.17 mW/cm²; 525 nm: 8.82 mW/cm²; 630 nm: 7.2 mW/cm²) over a course of up to 20 minutes. The fluorescence intensity was measured at t=0 minutes, prior to irradiation, and the normalized fluorescence intensity value was set to 100%. As shown, an increased light intensity leads to more efficient and faster bleaching. The acceleration effect tapers off as fluorophores decolorize. For the absolute difference in fluorophore intensity see the table next to each graph; these data confirm that photobleaching with stronger (i.e. higher irradiance) light accelerates dye decolorization initially. The stronger the irradiating light, the better the decolorization. All results are shown as the mean±SD of n=3.

FIG. 8A-8B. Influence of pH and buffer systems on SPS-mediated bleaching in vitro. Dyes were used at a concentration of 5 μM with 10 g/l sodium peroxydisulfate (42 mM) in different buffers (as indicated). Irradiation for purposes of photobleaching was performed for each dye with the optimal LED set to full power (470 nm: 13.17 mW/cm²; 525 nm: 8.82 mW/cm²; 590 nm: 2.52 mW/cm²; 630 nm: 7.2 mW/cm²) over a course of up to 20 minutes. The fluorescence intensity was measured at t=0 minutes, prior to irradiation, and the normalized fluorescence intensity value was set to 100%. (A) Influence of the pH value for a PBS buffer system. Bleaching was performed in PBS with pH values ranging between 6 and 8 (6, 7.2 and 8). Overall bleaching efficiency was not impacted by the buffer pH for all tested dyes. (B) Influence of different buffer systems. Bleaching efficiency was tested in PBS, TBS (both pH 7.2) and ddH₂O. Overall bleaching efficiency was not impacted by the buffer system for all tested dyes. All results are shown as the mean±SD of n=3.

FIG. 9. Comparing bleaching efficiency of different peroxydisulfate salts in vitro. Dyes were used at a concentration of 5 μM with 42 mM of a peroxydisulfate salt (ammonium peroxydisulfate (APS), sodium peroxydisulfate (SPS) or potassium peroxydisulfate (PPS)) in PBS. Irradiation for the purpose of photobleaching was performed for each dye with the optimal LED set to full power (470 nm: 13.17 mW/cm²; 525 nm: 8.82 mW/cm²; 590 nm: 2.52 mW/cm²; 630 nm: 7.2 mW/cm²) over a course of up to 20 minutes. The fluorescence intensity was measured at t=0 minutes, prior to irradiation, and the normalized fluorescence intensity value was set to 100%. No difference could be observed for the bleaching efficiency of different peroxydisulfate salts when used at the same concentration. All results are shown as the mean±SD of n=3.

FIG. 10. Effective SPS-mediated photobleaching of immunofluorescence signals in situ. A formalin-fixed paraffin-embedded (FFPE) human tonsil section was labeled with 3 target probes, including a nuclear stain (DNA-PI) and two antibodies (anti-panCK and anti-Histone H3). Each target probe was chemically conjugated to a different reporter probe/fluorophore (as indicated above each column). Reporter probes were excited and imaged with appropriate wavelengths to produce the images in the top row. Middle row: Irradiation with white light in PBS only (20 sec, 420-750 nm, 800 mW/cm²) resulted in partial weakening of the fluorescence signal (compared to Stain Image in Top row). Bottom row: Irradiation with white light in PBS+5 mg/mL SPS led to complete removal of fluorescent signals after 10 seconds. These data demonstrate that SPS is necessary for rapid and complete fluorophore decolorization in situ. Scale bar represents 100 μm.

FIG. 11. Effective SPS-mediated photobleaching of fluorescent RNA FISH signals in situ. Three different fluorescent molecules were removed using photobleaching+SPS. FFPE human tonsil was labeled with 3 RNA target probes, each of which was detected with a different fluorophore (as indicated above each column). Reporter probes were excited and imaged with appropriate wavelengths to produce the images in the top row. Middle row: Irradiation with white light in PBS only (20 sec, 420-750 nm, 800 mW/cm²) resulted in partial weakening of the fluorescence signal (compared to Stain Image in Top row). Bottom row: Irradiation with white light in PBS+5 mg/mL SPS led to complete removal of fluorescent signals after 10 seconds. These data demonstrate that SPS is necessary for rapid and complete RNA probe fluorophore decolorization in situ. Scale bar represents 100 μm.

FIG. 12A-12B. Selective SPS-dependent irradiation of fluorophores under selective wavelength illumination in situ (single wavelength). (A) Human FFPE tonsil tissue section that was stained with antibodies against CD45 and histone H3 labeled with two different fluorophores, AF488 and AF647, respectively. The image shows the overlay of the two labels in green and magenta. (B) Selective SPS-mediated photobleaching of fluorophores was performed via position-alternating exposure of the tissue to either 454-497 nm (60 mW/cm²) or 626-644 nm (40 mW/cm²) light for 10 s. This resulted in leftover signal in each position, where AF488 remained following irradiation with a 626-644 nm light, while the AF647 remained after irradiation with a 454-497 nm light. This demonstrates wavelength selective SPS-mediated fluorophore removal in situ.

FIG. 13A-13D. Repeated SPS-mediated photobleaching does not reduce the ability to detect biomarker signals in situ. Different 5 μm FFPE tissue sections were stained with Sytox Orange DNA dye, imaged and subsequently exposed to 50 cycles of SPS-mediated photobleaching (532-555 nm, 100 mW/cm² for 10 s), with repeated DNA staining & imaging every 5 cycles to assess tissue integrity. After 50 cycles of photobleaching, all sections were stained with fluorescent-labeled antibodies against CD20, FoxP3, pan-CK, and CD3, and then re-imaged. As controls, serial sections of the same tissues were stained with a DNA stain and antibodies, but without the 50 repeated photobleaching cycles. (A) Tonsil section (B) thymus section (C) breast cancer section (D) head & neck cancer section. Image panels show stained serial tissue sections that underwent 50× cycles of enhanced photobleaching in the top row versus no treatment in the bottom row. The results show that after 50 cycles of repeated SPS-enhanced photobleaching, all markers could be detected on all tested tissue sections, suggesting that epitope stability is preserved through at least 50 cycles of SPS-enhanced photobleaching under the stated conditions.

FIG. 14A-14C. SPS-mediated photobleaching does not induce degradation of tissue structure after repeated exposures in situ. Cellular DNA of 5 μm FFPE tissue sections was stained and imaged before the first cycle and after 50 cycles of repeated enhanced photobleaching (as described in FIG. 13) to evaluate the effect of SPS-mediated photobleaching on tissue integrity. (A) Normal tissue samples; (B) Cancer tissue samples. (C) Number of detected cells by StarDist segmentation algorithm-based cell quantification before and after 50 cycles of repeated SPS photobleaching. The consistent unaltered shape, staining pattern & intensity of nuclei between the two experimental conditions indicate that there is no qualitatively measurable damage to the tissue structure. Tissue integrity thus remains intact even after repeated episodes of enhanced photobleaching. This is also reflected by an average difference of under 3% in the number of detected cells between cycle 1 and cycle 50 across all samples (C). The merged images of cycle 1 and 50 data shown in the bottom row of (A) and (B), indicate no differences in the fine morphology of nuclei compared across conditions, ultimately confirming preservation of tissue morphology after SPS-enhanced photobleaching.

FIG. 15A-15B. SPS-mediated photobleaching leads to measurable reduction in endogenous tissue background autofluorescence. Tissue autofluorescence was imaged on two serial 5 μm pancreas adenocarcinoma FFPE sections in different optical excitation channels to capture the broad range of autofluorescence emission spectra. One section was subjected to 10 s of SPS-mediated photobleaching with white light (420-750 nm, 800 mW/cm²), while a control section was not bleached prior to autofluorescence imaging. Both sections were subsequently stained with antibodies against Ki-67 and pan-CK as well as Sytox Orange DNA dye. (A) After StarDist cell segmentation based on the DNA stain, the pre-bleaching average cell autofluorescence intensity was measured in segmented regions of the sections. In the green (507-527-nm) and yellow (558-586 nm) range of the autofluorescence emission spectrum, the tissue autofluorescence was reduced by a factor of approximately 4 and approximately 4.5, respectively, after 10 s of enhanced photobleaching. In the orange (612-644 nm) and red (672-712 nm) range the autofluorescence reduction was by a factor of ˜2.8 and ˜1.6, respectively. (B) The staining of both Ki-67 and pan-CK markers is easier to distinguish from the tissue autofluorescence in the sections that underwent enhanced photobleaching (for better representation, some stained cells are marked with an arrow). This demonstrates that enhanced photobleaching increases data quality by decreasing background autofluorescence in fluorescence microscopy.

FIG. 16A-16C. Chemical bleaching of fluorophores with LiBH₄ and H₂O₂, but not SPS, leads to the formation of gas bubbles in enclosed microfluidic systems. (A) A typical microfluidic sample chamber used for multiplex imaging is shown after a 15 min bleaching reaction with 5 mg/ml SPS in ddH2O; no gas bubbles are formed within the fluidic chamber or near the tissue sample that is mounted in the middle of the chamber (arrow). (B) Release of hydrogen gas during incubation of the sample with 1 mg/ml LiBH₄ in ddH2O for 15 min causes excessive bubble formation within the enclosed microfluidic chamber. The formed bubbles are difficult to remove from the chamber, impede optical imaging and may damage the sample and/or compromise chamber integrity due to pressure buildup and displacement of liquid. (C) A microfluidic sample chamber is shown after a 30 min bleaching reaction with 3% H₂O₂ in 20 mM NaOH. The production of oxygen gas leads to the formation of ubiquitous bubbles within the enclosed microfluidic system, affecting optical imaging and chamber integrity.

FIG. 17. Storage stability of SPS solution. SPS was dissolved in PBS buffer to a final concentration of 5 mg/ml and stored at 4° C. for 12 months. AF488 dye was mixed with stored and freshly prepared SPS solution to a final dye concentration of 5 μM and exposed to low intensity white light (LED 420-700 nm; 25.2 mW/cm²) for 10 minutes. Before and after the white light exposure, the fluorescence of the SPS/dye solution was measured on a DeNovix DS-11 with a FX module to determine bleaching effectiveness. Under these conditions, freshly prepared SPS solution reduced AF488 dye fluorescence by 65% compared to non-irradiated solution. Similarly, SPS solution stored for 12 months at 4° C. reduced dye fluorescence by 63% compared to the non-irradiated solution. This indicates comparable bleaching efficiency to fresh solution, with no significant loss of SPS reactivity during storage under the described conditions. Results are shown as the mean±SD of n=3.

### DETAILED DESCRIPTION

### Introduction

Various methods are used in biomedical research to label biomarker targets in a biological sample. For example, protein targets may be detected in tissue sections via histochemistry, immunohistochemistry (IHC), or immunofluorescence imaging (IF). RNA and other gene targets may be detected via in-situ hybridizations (ISH), fluorescent in-situ hybridizations or single-molecule imaging. Most of these methods have been developed around optical microscopy and are intended to image the location of the biomarker target with high optical resolution.

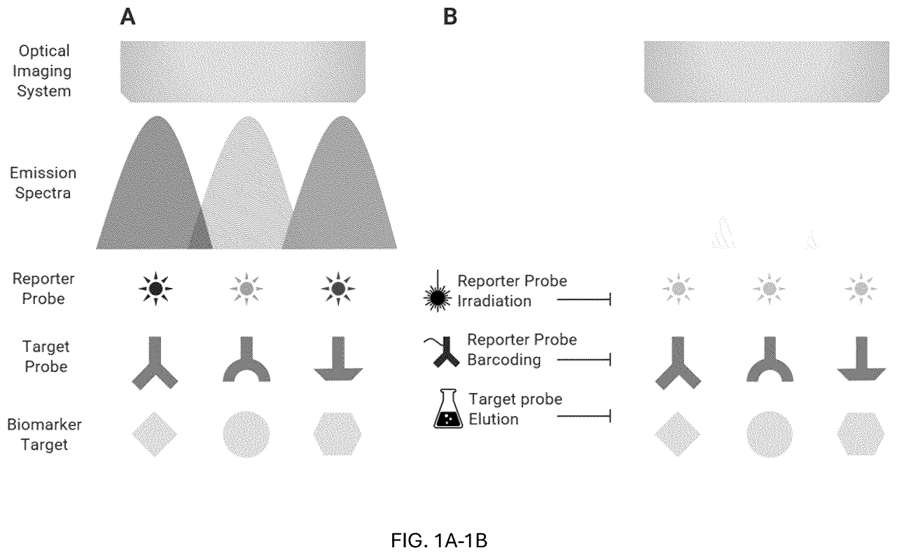

Imaging-based biomarker detection has traditionally been limited by the number of biomarkers that can be simultaneously imaged from a single sample. For example, 3,3′-diaminobenzidine (DAB) is a widely used chromogen in IHC and it produces a brown precipitate (reporter signal) that can be imaged with a conventional light microscope. DAB has excellent detection sensitivity, but it has limited use for multiplex imaging and is rarely used to detect >1 biomarkers (maximum 2-3). In comparison, IF staining methods offer greater flexibility to multiplex in situ. These methods employ organic fluorescent dyes (reporter probes), which can be selectively excited to emit fluorescent reporter signals within defined spectra (e.g. red, green, blue in FIG. 1A). Each signal can be used to selectively identify a target probe, and each signal is separately collected with conventional epifluorescence optics. Via these means it is possible to collect optical signals from ≤5 biomarkers in a single tissue without modifications to standard workflows.

Recent years have seen explosive demand for the imaging-based detection of even more biomarkers in-situ. For the above-mentioned approaches, IHC and IF, this would require experimenters to use additional biological samples from the same source (e.g. a serial histological section) and to repeat multiplex staining several times. However, this approach limits the experimenters' ability to accurately determine the co-expression and interactions of all their targets at the single-cell level. An added complication exists with the incompatibility of certain widely used approaches with one another. Specifically, chromogenic DAB staining and IF employ the same target probes (antibodies) to detect the same biomarkers, however the detection probes are different, and there are few practical workflows that allow integrations of these approaches onto single samples.

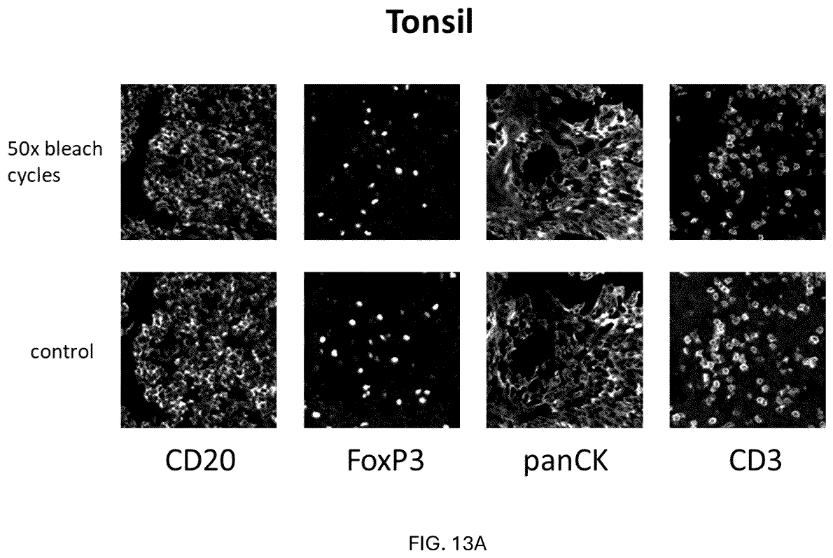

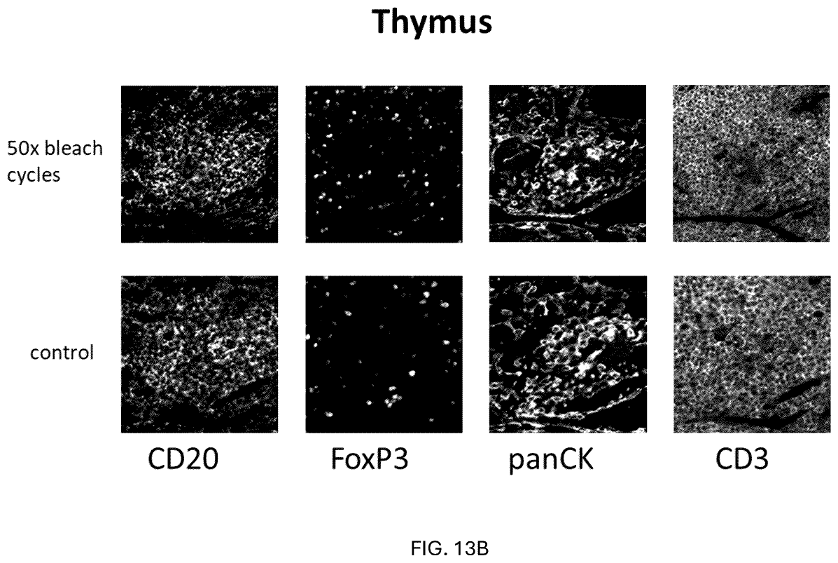

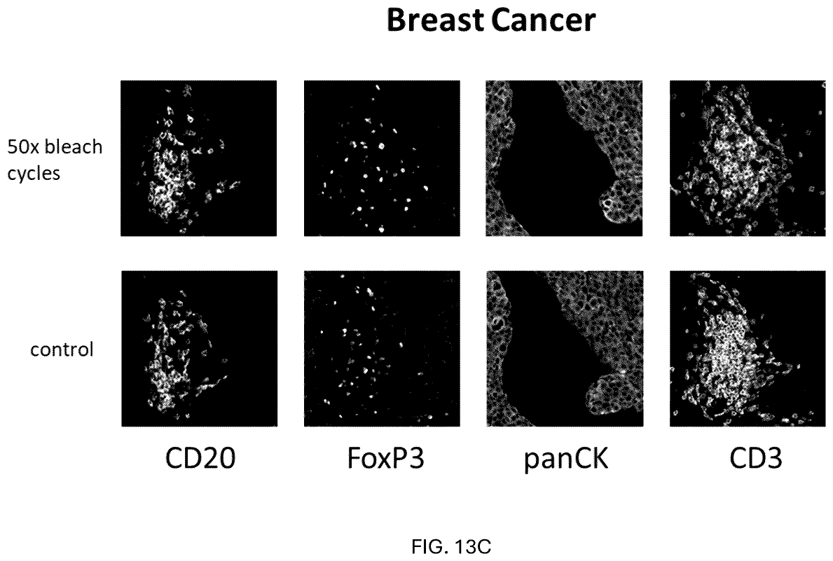

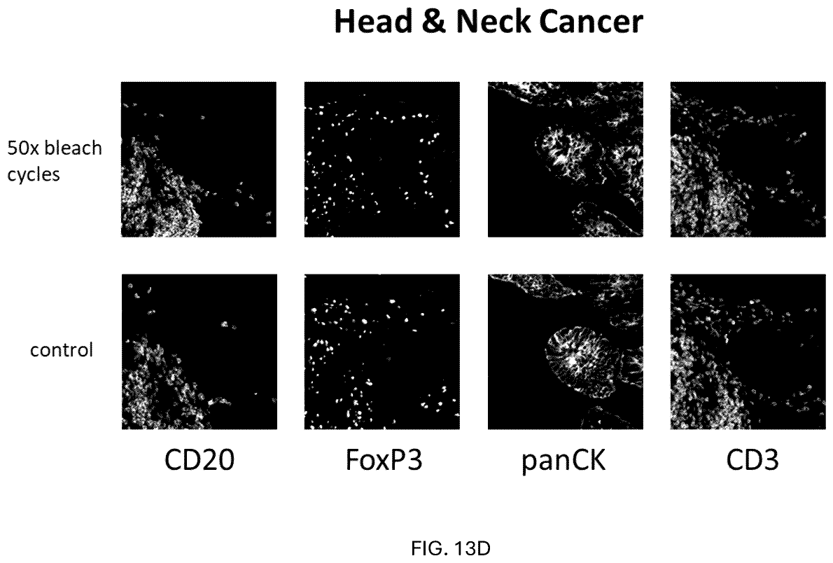

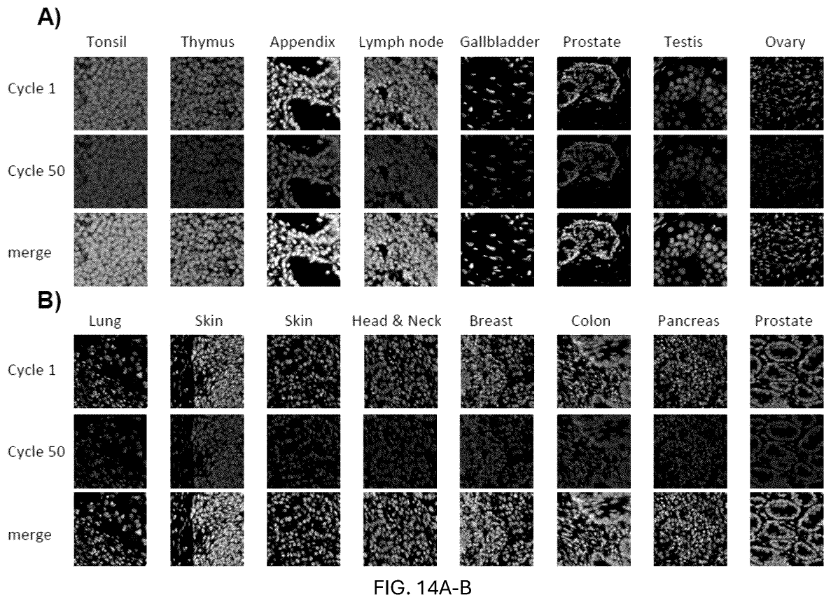

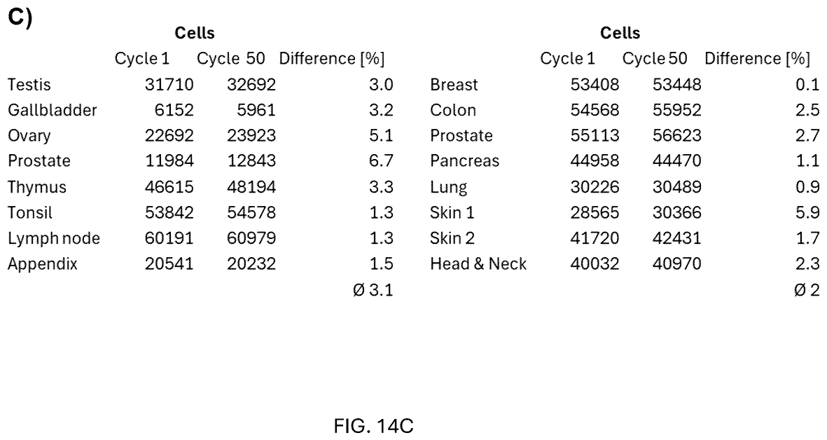

FIG. 1B provides a schematic overview of multiplex imaging technologies that have been developed to scale biomarker plexity—i.e. the number of target probes that can be selectively imaged from a single specimen. In their basic configuration, these techniques resemble standard epifluorescence microscopy: they deploy 2-5 target probes that are each labeled with a spectrally distinct reporter probe. Each probe is then selectively excited and imaged and thereby used to represent a biomarker of interest. Increased plexity of targets is then achieved through an iterative process, where (1) biomarkers are labeled with a target probe—(2) the target probe is labeled with a reporter probe—(3) the reporter probe is imaged with an optical microscope—(4) the target or reporter probe is removed and/or inactivated. This process is repeated n times, until all biomarkers of interest have been imaged (e.g., see FIGS. 13-14, showing results for n=50 cycles with 4 target probes (antibodies) each, ultimately resulting in a total readout from 200 biomarkers). The main difference between iterative workflows relates to how reporter probes are designed and how they are removed from the tissue and/or inactivated whilst remaining on the tissue. These strategies are discussed in more detail below.

### Irradiation (Photobleaching) of Reporter Probe Signals

Irradiation or photobleaching of reporter probes is arguably the simplest probe removal method used in iterative multiplex imaging (FIG. 1B). In this approach, multiple target probes are directly or indirectly labeled with fluorescent reporter probes. After imaging, the reporter probes are decolorized via irradiation; this results in a loss of the reporter probe signal, but the reporter probe itself remains within the tissue, as does the target probe. Irradiation thus leads to reporter probe inactivation rather than probe removal.

Photobleaching may be accomplished without any chemical additive in a normal physiological buffer (e.g. PBS). This strategy is advantageous because it produces minimal damage to the tissue (depending on the wavelength and intensity of light employed). Moreover, fluorescent antibodies are readily available from commercial suppliers, and it is relatively easy to source the reagents needed for multiplex imaging experiments. Yet, photobleaching can be time consuming as many fluorophores are quite photostable and it may not proceed to completion; this ultimately results in reduced signal-to-noise ratios and non-selective imaging data². In addition, continued exposure of tissue samples to long irradiation steps may cause cumulative damage to the biological sample and hinder further detection of targets.

Multiple strategies have therefore been developed to enhance photobleaching efficiency via the addition of chemical agents. The Cyclic ImmunoFluorescence (CyCIF) workflow, for example, combines H₂O₂ with irradiation to oxidize/deactivate fluorescent probes and reduce tissue background fluorescence in multiplexed imaging experiments. The method is well-established and yields good results, but it is slow and may take several days to complete³. The reaction that is associated with the exposure of tissues and probes to H₂O₂ furthermore leads to the formation of oxygen gas, which is problematic in enclosed automated experimental systems that are nowadays deployed in many research labs (e.g., CellScape™, PhenoCycler™, CosMxx, Merscope™ systems). Moreover, H₂O₂-mediated oxidization of reporter probes is not spatially restricted and may have the side effect of damaging/changing certain epitopes.

Another chemical photobleaching approach has been published in WO 2014/093455. In this system, organic borate salts are activated by visible light to chemically deactivate fluorescent probes. While the removal of the fluorescence signal is effective, several drawbacks limit the usability of this system. First, the protocol requires the introduction of reactive oxygen scavengers, as without them there is severe epitope damage, which prevents iterative detection of biomarkers. Second, as organic borate salts have low water solubility, more complex borate compounds with higher water solubility must be designed. While having better properties, these compounds are not easily accessible nor are they commercially available. Lastly, the reagents needed to accomplish this workflow have poor storage properties, all of which make the commercial development and deployment of this system impractical.

### Chemical Removal of Reporter Probe Signals or Target/Reporter Probe Complexes

Assays that deploy chemical and/or thermal means to remove reporter probe signals and/or entire target-reporter probe complexes are widespread in the multiplex imaging community (FIG. 1B). Collectively referred to as ‘stripping’ methods, these approaches serve the same general purpose of target probe- or reporter signal removal, but they do so with varied efficiencies. A clear advantage of stripping methods is that they offer flexibility for use with multiple histological techniques, and they generally do not require a priori modifications to target probes, reporter probes or tissue specimens. A drawback of all or most of these techniques is that they inflict some degree of damage to the tissue specimen, which ultimately limits the degree of plexity that can be achieved. Reviewed below are certain approaches that seem to inflict only minor/modest damage and have thus been adopted widely for research aimed at multiplex imaging with medium to high plexity.

Antibody elution results in the removal of target-reporter probe complexes and is achieved by administering a chaotropic buffer of low pH (2.0) with sodium dodecyl sulfate (SDS), and reducing agents (DTT, TCEP, etc.). The removal of the target/reporter probe complex is rapid and can be achieved within 2 minutes at 50° C. This allows for efficient completion of iterative multiplexing experiments⁴. However, repeated exposures to acidic, chaotropic buffers and high temperature may affect tissue integrity and hence the ability to achieve highly multiplexed imaging data⁵.

Another chemical signal removal method, iterative bleaching extends multiplexity (IBEX), employs lithium borohydride (LiBH₄) to achieve multiplexing in fixed-frozen and formalin fixed tissue samples. IBEX generally achieves good results, and its open concept makes it useful for direct and indirect immunofluorescence applications alike⁶. However, borohydrides are highly toxic compounds and require special handling. In addition, LiBH₄ reacts with water when dissolved which then produces hydrogen gas (e.g. 1 g LiBH₄ produces >4 liters of hydrogen gas); this leads to excessive bubble formation, which in turn makes the deployment of IBEX in an enclosed system difficult and potentially hazardous. Lastly, the low stability of borohydrides in solution limits the commercial production, storage and deployment of reagents that are needed for the IBEX protocol.

Another recently published method of cyclic signal generation and removal, spatial photo-inactivation enhanced cyclic target resolved multiplexing (SPECTRE-Plex), utilizes meta-chloroperoxybenzoic acid (m-CPBA) as a chemical oxidizer to inactivate fluorescent reporter probes on a sample⁷. This method is faster than LiBH₄ or H₂O₂-based signal removal, and the method does not lead to the formation of gas during the process, which would allow it to be deployed in an enclosed fluidics system. However, this method, like most chemical oxidation processes, is non-selective and will react with all probes and the biological target tissue indiscriminately, which can lead to damage and alteration of the biological substrate. It is also not effective against certain reporter dyes, like AlexaFluor 594, which are commonly deployed in immunofluorescence imaging. The compound is more stable in an aqueous working solution than LiBH₄ or H₂O₂, but with a shelf life of less than 36 h it still requires regular preparation of fresh working solutions, which might result in experimenter errors and certainly requires more hands-on time.

### Antibody Barcoding

Antibody barcoding has gained momentum as another method to achieve high-plex biomarker imaging in-situ. For example, CO-Detection by IndEXing (CODEX) employs an oligo barcoding strategy, in which distinct oligo barcodes are attached to the target antibodies (target probes). Once used to label biomarker targets in a tissue, each type of antibody can be tagged with a complementary oligo-barcoded fluorescent reporter probe. After imaging, the probes are removed via de-hybridization or dissolution from the antibody-oligo complex, and a new cycle of probe labeling can be started. With this method, spectrally compatible probes can be delivered and imaged iteratively in small groups on a microscope with conventional epifluorescence optics⁸. The method is isothermal and does not damage tissues and is currently regarded as the most well-suited system to scale the plexity of biomarker imaging experiments as far as >100 targets in a sample⁹.

### Drawbacks of Elution and Barcoding Techniques

While powerful and well-established, elution and barcoding approaches have limitations that are becoming increasingly obstructive to the design and adoption of multiplexing experiments. Conjugating oligo-barcodes to antibodies, for example, may disrupt the cysteine bridges in antibodies, which in turn affects the ability of an antibody to detect appropriate target epitopes⁸. Oligo barcodes may also non-specifically hybridize with genomic DNA in the tissue sample, which leads to increased detection of nonspecific nuclear staining signals as well as an overall decreased signal to noise ratio. In addition, reporter oligos are in themselves variable and they do not always produce adequate optical signals¹⁰. On the other hand, elution-based multiplexing approaches require buffers with acidic pH as well as repeated heating of tissue samples, and this has been shown to affect tissue integrity. Deen et al 2023, reported distorted cell nuclei and failure of membrane markers in fresh frozen tissue samples that were multiplexed with a well-established elution method¹¹. Even if successful, elution protocols require extensive optimization, and they are rarely used or successful if an investigator seeks to image more than a few dozen biomarker targets¹⁰. Evidently, both approaches limit the degree to which multiplexed biomarker imaging can be performed and the development of new systems that do not suffer from these drawbacks is justified. The present disclosure describes a hybrid system of irradiation and chemical reporter probe inactivation, termed herein enhanced photobleaching.

The present technology is grounded in the principle of reporter probe irradiation for the purpose of photobleaching but achieves superior results via chemical enhancement of the signal removal process, while avoiding the drawbacks of well-known photobleaching methods.

### Enhanced Photobleaching Methods and Compositions

Disclosed herein are methods, systems, platforms, kits, and compositions useful for processing biological samples, to image and visualize targets in the sample, and to more efficiently and accurately perform iterative, multiplexed, or repeated imaging and visualization of targets.

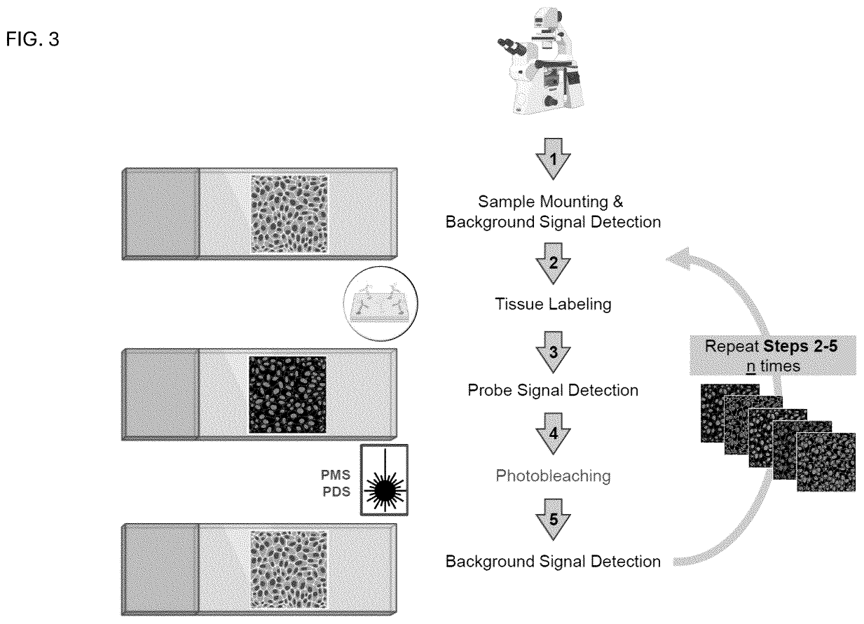

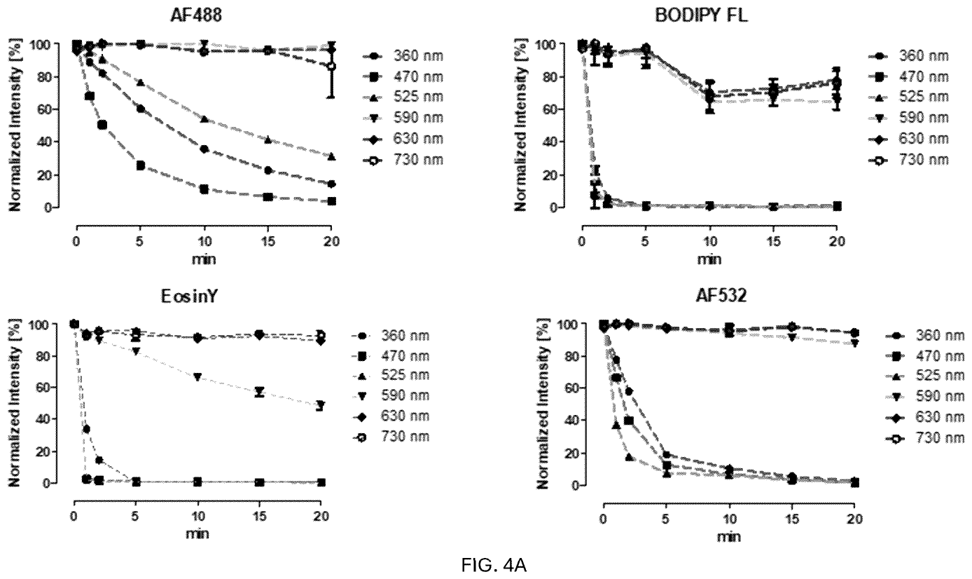

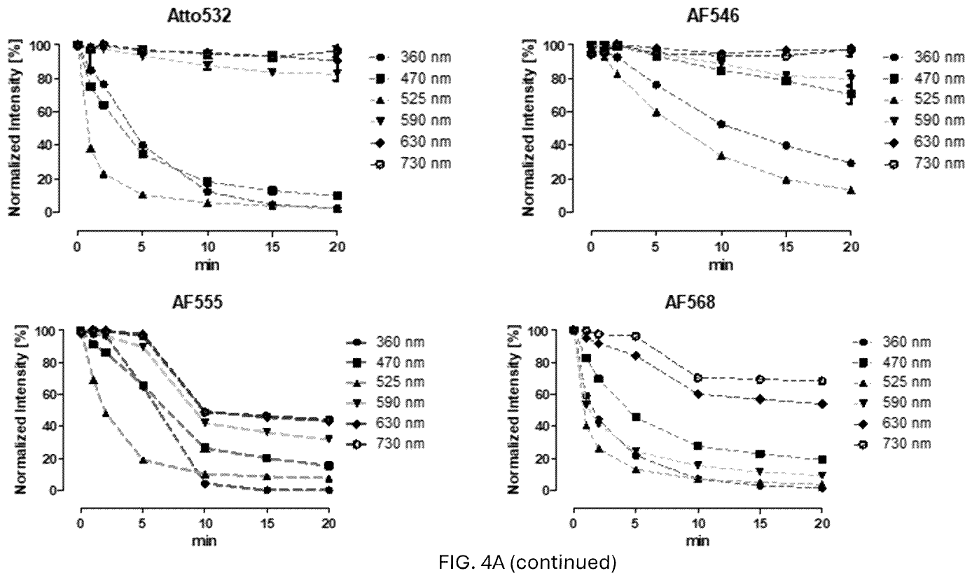

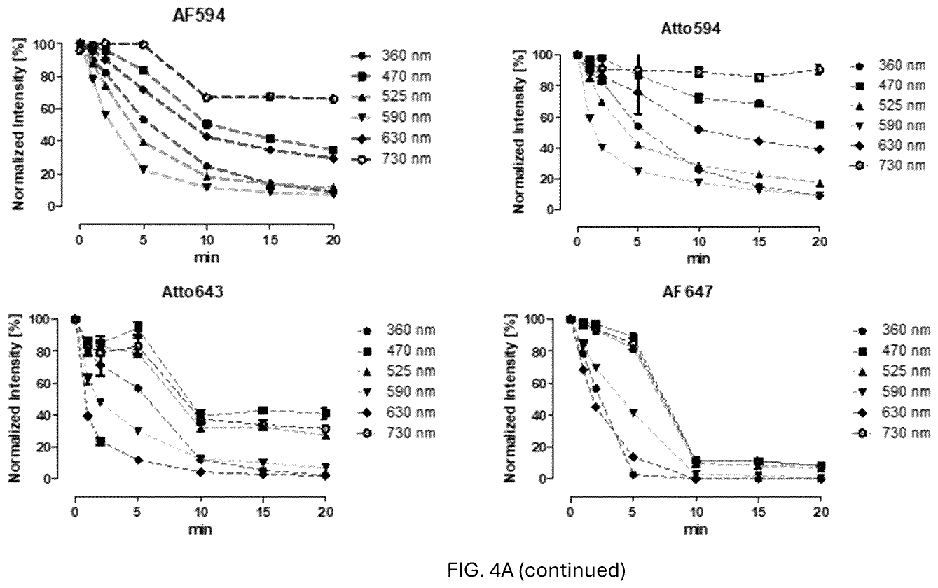

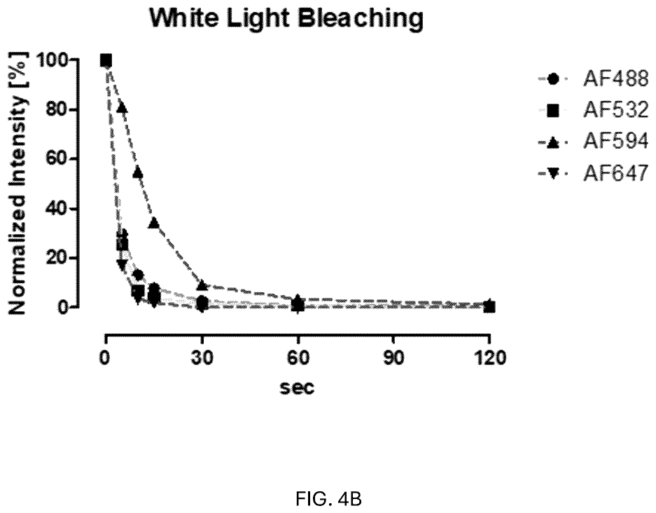

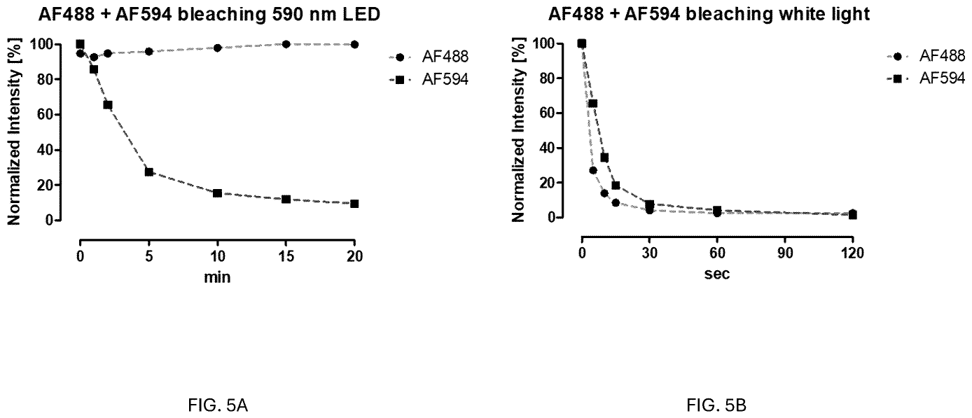

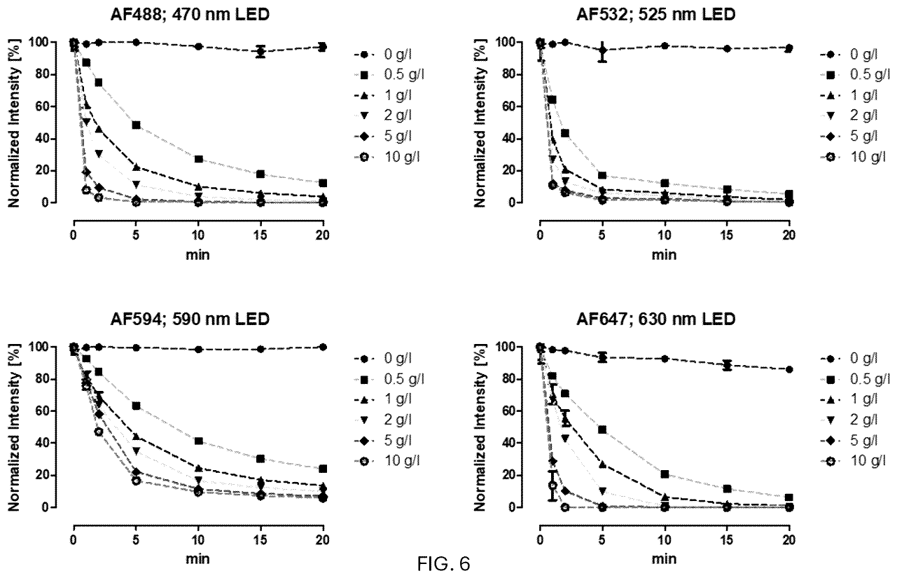

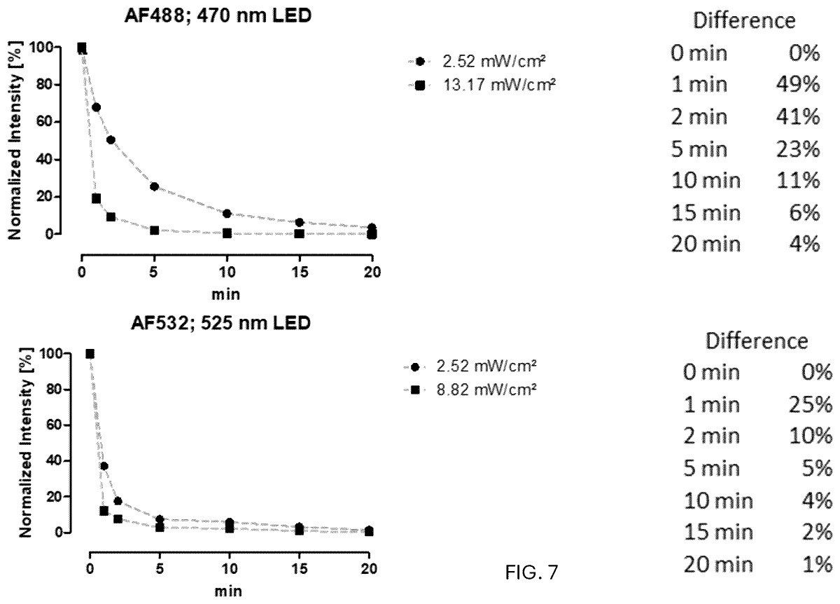

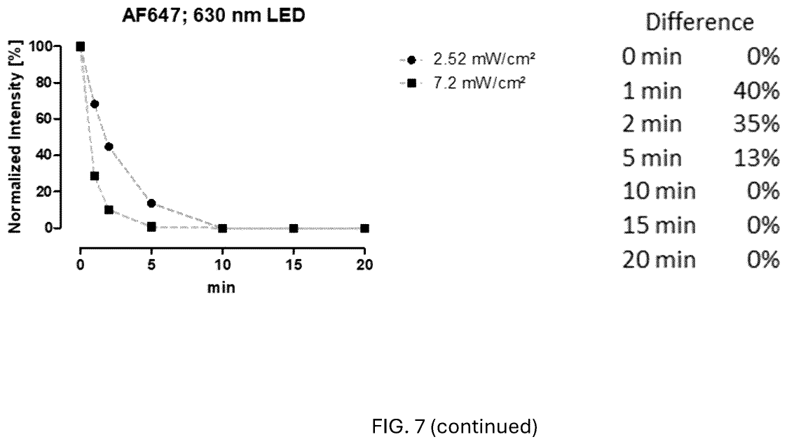

The disclosed technology, termed “photobleaching” or “enhanced photobleaching” herein, is depicted diagrammatically in FIG. 3 as it relates to use in multiplex imaging experiments. The technology of the present disclosure encompasses both general photobleaching (e.g., irradiating a labeled sample using white light) and selective photobleaching. “Selective photobleaching” or “selective bleaching” as used herein refers to targeted enhanced photobleaching, using a selected wavelength range of light, which does not include white light to irradiate the sample. In some embodiments, the selected wavelength range of light comprises the entire wavelength range of light useful to detect a detectable label, such as a fluorescent label or a dye. In some embodiments, the selected wavelength range is within the wavelength range required to detect the detectable label (e.g., the range is narrower than the full range useful to detect the detectable label). In some embodiments, the selected wavelength range comprises overlapping wavelength ranges, or different wavelength ranges, e.g., to selectively photobleach multiple detectable labels simultaneously and/or sequentially. In some embodiments, a sample may be labeled, and selectively photobleached (e.g., with a selected wavelength range, one or more times) and then generally photobleached with white light to eliminate any remaining signal. In some embodiments, a sample may be photobleached with white light to reduce or remove any signal (e.g., signal remaining from previous experiments performed on the same sample, or to reduce background, etc.), and then may be labeled, imaged, and then selectively photobleached.

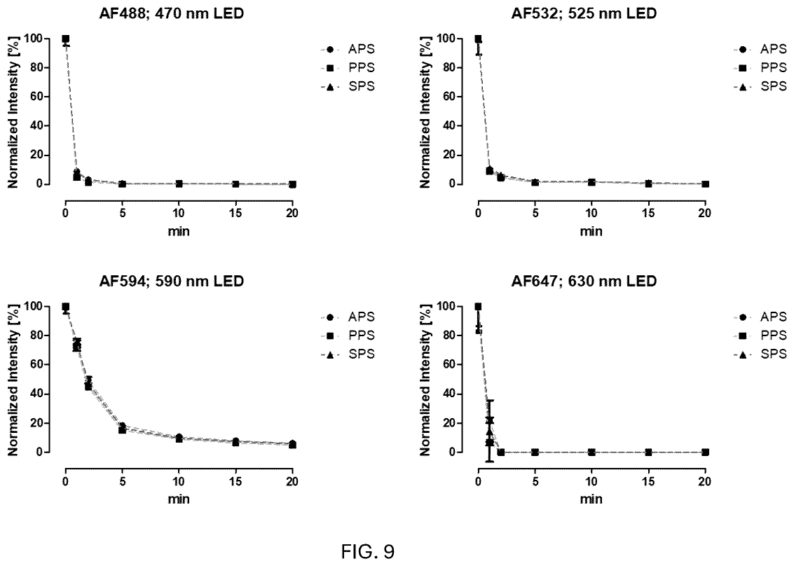

Peroxydisulfate (PDS, or termed interchangeably, sodium persulfate, SPS) and peroxymonosulfate (PMS) are radical initiators that can be activated to produce the strongly oxidizing SO₄·⁻ radical and they find widespread use in the remediation of water and soil by degrading organic contaminants¹²⁻¹⁵. The structures of PMS and PDS are provided in their salt forms, respectively, below.

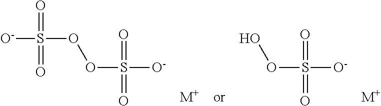

wherein M+ is an organic or inorganic cation. While sodium peroxydisulfate (also termed “sodium persulfate” (SPS)) is used in the examples, other cationic forms of PDS or PMS will also work to decolorize organic dyes as demonstrated in FIG. 9 (e.g., potassium and ammonium peroxydisulfates).

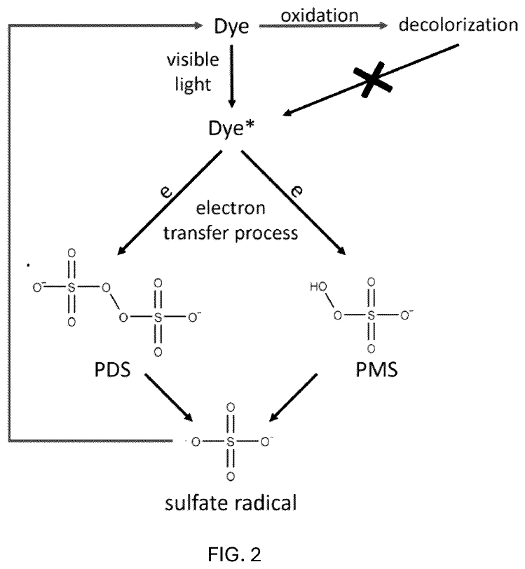

Activation of PDS/PMS is mostly done via heat, UV irradiation (<300 nm) and inorganic catalysts (e.g. transition metals), but the activation by visible light has thus far received little attention. Gao et al. 2017 reported that visible-light excited organic fluorescent dyes can act as photosensitizers to activate PDS/PMS¹. The mechanism involves excitation of the dye from its ground state by visible light followed by an electron transfer process from the excited dye to PDS/PMS, ultimately leading to activation of the molecules and the production of sulfate radicals. In this system the dye does not only act as photosensitizer but also as the substrate for the sulfate radicals, which oxidize and decolorize the dye after first being activated by it (FIG. 2). Ultimately, this process may allow the selective decolorization of fluorescent dyes by PDS/PMS under visible light irradiation and could thus pose an attractive probe removal solution for multiplexed imaging assays.

This present technology relates to an irradiation/photobleaching method that exploits PDS and PMS as enhancers of the photobleaching process. In contrast to Gao et al. 2017, which discloses general photobleaching of several potentially harmful compounds in environmental waste, the present technology provides specific, targeted photobleaching of detectable labels in a biological sample. The photobleaching process of the present technology provides precision and specificity to the process, allowing sample preservation and cost effective, efficient multiplexing with iterative and sequential target detection. Thus, the present technology will provide numerous benefits and find widespread use in multiplexing applications, overcoming shortcomings of other currently available methods.

The methods, systems, platforms, kits, and compositions disclosed herein comprise one or more of the following characteristics:
1) are based on enhanced photobleaching as laid out in FIG. 2;
2) require only reagents that are commercially available, stable, non-toxic and of good solubility (e.g. PMS and PDS);
3) do not affect tissue integrity (e.g. via repeated exposures of tissues to acidic pH and elevated temperature);
4) do not require alterations to target probes (e.g., by conjugating the antibodies to oligonucleotides) or tissues (e.g. elution via repeated exposures of tissues to acidic pH and elevated temperature);
5) are reasonably fast;
6) are compatible with a wide range of reporter probes (e.g. rhodamine, cyanine and BODIPY dyes), as well as other general tissue stains (eosin, and other dyes);
7) are compatible with and allow the integration of different workflows that are based on optical imaging for signal detection;
8) are compatible with common hardware designs (e.g. closed microfluidic systems); and
9) are attainable with illumination settings offered by standard epifluorescence imaging technologies.

The photobleaching technology disclosed herein, as it relates to multiplex imaging experiments, is depicted diagrammatically in FIG. 3, and is discussed in more detail below.

Disclosed herein are methods and compositions useful for the detection of targets in a biological sample. In embodiments, a biological sample is processed for imaging to detect a single target, a single set of targets simultaneously, or to detect multiple targets or sets of targets sequentially.

Sources of the biological sample are typically from a multicellular subject, such as a plant, fungus, or an animal (e.g., mammal, bird, reptile, fish, amphibian), but may also be from a single cell organism, such as a bacteria, yeast, or protist.

Biological samples that are amenable to the present technology include but are not limited to tissue samples, liquid samples that include one or more cells (e.g., such as a blood, urine, bone marrow sample), and cells or tissue in culture (e.g., organoids or tissue explants).

A biological sample such as a tissue sample may be fresh or it may be preserved (e.g. frozen, formalin fixed, paraffin embedded), and/or fixed to a solid support such as but not limited to a slide, bead, microwell, multiwell plate, nitrocellulose, PDVF membrane, flow cell, or paper. In embodiments, fresh or preserved samples may be configured on a solid support for analysis and imaging according to the methods disclosed herein. In some embodiments, the biological sample is configured in an enclosed microfluidic system for reagent delivery onto the sample.

Exemplary non-limiting tissue samples include biopsy samples, surgical resections, and surgical aspirates. Exemplary non-limiting liquid biological samples include blood, bile, bone marrow aspirate, breast milk, cerebral spinal fluid, plasma, saliva, semen, serum, sputum, stool, swabs (oral, nasal, vaginal fluids), synovial fluid, and urine.

In embodiments, target molecules include molecules such as proteins, nucleic acids, carbohydrates, lipids, and combinations thereof. Such molecules may comprise all natural components, or may comprise one or more non-natural components (e.g., modified amino acids, nucleic acids, chemical moieties, etc.). Methods for detecting such molecules are well known in the art, and the design and use of appropriate detection probes (e.g., antibodies, fragments thereof, nanobodies, nucleic acids, aptamers, etc.) to specifically bind to such molecules is not the focus of the present technology. Rather, the present technology is directed to the quenching or selective extinction of one or more signals generated by a signal generator associated with the detection probes, i.e., the reporter probes used to detect any of the aforementioned molecules.

For example, numerous methods of protein detection involve the use of a protein binding moiety (such as an antibody or fragment thereof), linked to or associated with a detectable label, (e.g., by contacting the binding moiety with a secondary molecule comprising a detectable label). Likewise, methods of nucleic acid detection typically involve a nucleic acid binding moiety (such as a probe which is complementary to all or a portion of a target nucleic acid), which is linked to or associated with a detectable label.

As used herein, the term “target probe” refers to a molecule able to specifically bind to a target molecule. Exemplary non-limiting target probes include antibodies, nucleic acids, aptamers, etc. Target probes may themselves include a detectable label or may be contacted by a secondary probe or tertiary probe, e.g., a “reporter probe” which comprises a detectable label.

As used herein, a “reporter probe” comprises a molecule including an optical signal generator (a detectable label). By way of example but not by way of limitation, a reporter probe may comprise an antibody, nanobody, a nucleic acid, etc. chemically linked to a detectable label.

As used herein the terms “reporter molecule,” “detectable label,” and “optical signal generator” are used interchangeably and refer to the molecule that provides the visualization signal. By way of example but not by way of limitation, in some embodiments, the detectable labels are dyes or are tissue/cell stains.

In embodiments, the signal generator is a dye expressed by the cell or organism (e.g., is genetically encoded). Exemplary non-limiting dyes include fluorescent proteins such as GFP, YFP, RFP, tdTomato, etc.

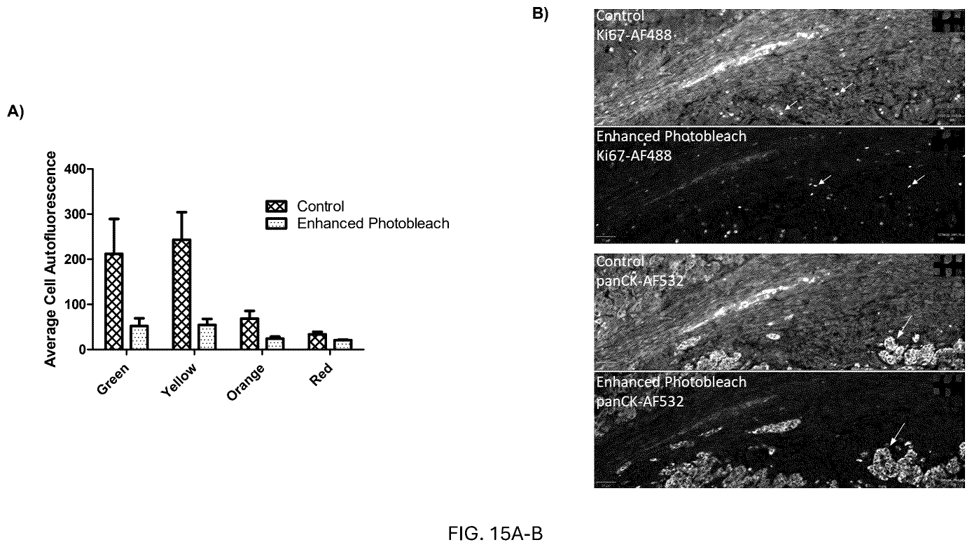

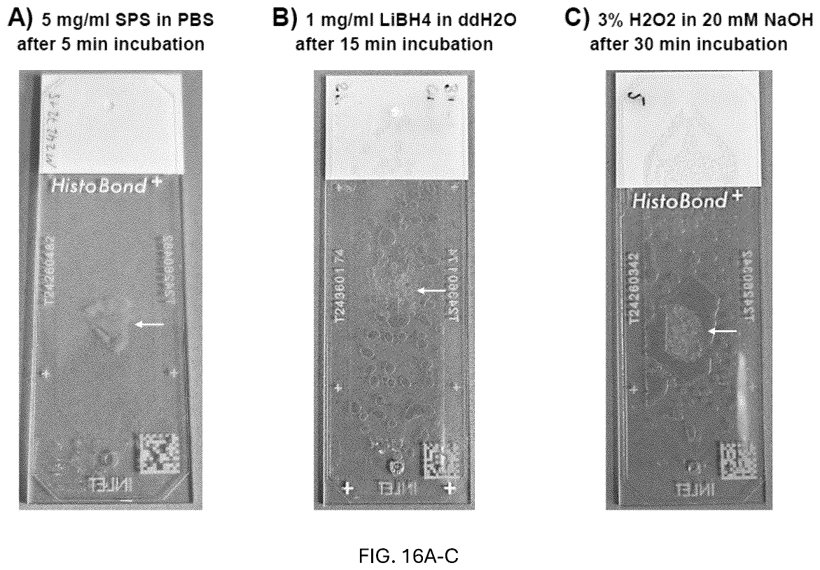

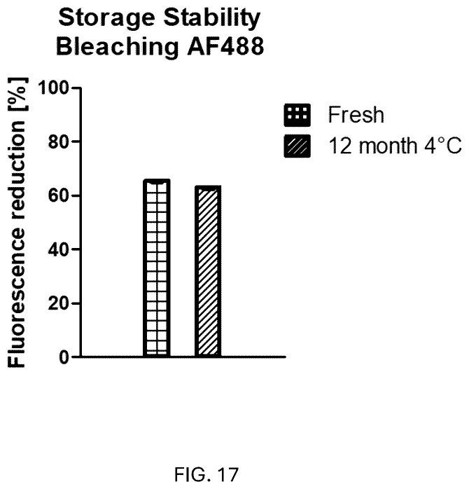

Tissue autofluorescence, which is not a dye per se, is a source of fluorescence ‘noise’ in tissue imaging applications (e.g. see FIG. 15).

In some embodiments, a detectable label comprises a stain (e.g., a general tissue stain or general cell stain) that allows for visualization of a cell or tissue of a biological sample. As is known in the art, stains highlight different features and/or components of a cell or tissue. By way of example, but not by way of limitation, two of the most common stains are hematoxylin and eosin. Hematoxylin is a basic dye that stains acidic molecules like DNA/RNA a blue color, while eosin stains proteins in a pink color. Sometimes these stains are used in combination and can be referred to as H&E staining. Both stains, singly or in combination, are useful in the context of the present disclosure. Additional, non-limiting stains include: mucin stains for the detection and visualization of mucopolysacchairdes (e.g., Alcain blue, Mucicarmine, period acid-Schiff (PAS); melanin stains, for dying and visualizing melanin (e.g., Fontana-Masson); and trichome stains for lipid visualization, (e.g., Gomori trichome, Mallory trichome, Sudan stains such as Red Oil O). Table 1 below provides exemplary, non-limiting stains that could be used in combination with photobleaching technology of the present disclosure. While some of the stains listed in Table 1 may not be amenable to selective photobleaching or may not be photobleached at all using the compounds and methods disclosed herein, such dyes may still be used in combination with other detectable labels that can be photobleached.

TABLE 1
Exemplary Stains

| Name of Stain | Comments |
|---|---|
| Alcian blue | Blue; common mucin stain |
| Aldehyde fuchsin | Purple/black; used to stain beta cells in the pancreas |
| Alkaline phosphatase | Red/blue; used for endothelial tissue |
| Bielshowsky stain | Black; used for neural plaques and tangles |
| Congo red | Red; typical for staining amyloid fibers |
| Crystal violet | Violet; can stain glia and neurons |
| Eosin | Pink/orange/red; typical for general staining when combined with |
| | haematoxylin |
| Fontana-Masson | Black/pink or red; stains melanin |
| Giemsa | Blue/violet/pink; commonly used in blood or bone marrow smears |
| Hematoxylin | Blue/purple; standard for general staining when combined with |
| | eosin |
| Luna stain | Purple/black; can stain mast cells and elastin |
| Nissl | Blue; stains the rough endoplasmic reticulum in neurons |
| Period Acid Schiff | Red/magenta; used to stain glycogen, basement |
| (PAS) | membranes, reticular fibers, cartilage, glycoproteins, glycolipids |
| | and mucins in tissues |
| Red Oil 3 | Red; used to stain fat emboli |
| Reticulin stain | Blue/black; stains reticular fibers |
| Sudan black | Brown-black; stains myelin tissue |
| Toluidine blue | Blue; stains mast cell granules |
| van Gieson | Red/blue/yellow; used to study blood vessels and skin, can |
| | stain collagen, nucleus, red blood cells, cytoplasm |

Detectable labels (a term used interchangeably herein with “optical signal generators”) are well known in the art. By way of example but not by way of limitation, detectable labels include dyes such as fluorophores of the rhodamine, cyanine and BODIPY dye families. Exemplary dyes include AF488, AF532, AF594, AF647, Atto532, AF546, AF555, AF568, Atto594 and Atto643.

As used herein, the term “fluorophore,” “fluorescent signal generator” and like terms refer to a chemical compound, which when excited by exposure to a particular wavelength of light, emits light at a different wavelength. Fluorophores may be described in terms of their emission profile, or “color.”

As is known in the art, different detectable labels such as fluorophores are useful in multiplexing assays, at least because different fluorophores can be visualized as different colors.

Table 2 summarizes dyes that are commonly used for fluorescent optical imaging applications (non-exhaustively). It also lists excitation wavelengths that can be generated with microscope optics or custom light sources. The excitation wavelength for each dye is used to promote emission at another wavelength, and this second wavelength emission is the signal that eventually ends up being captured as an image. In this capacity the dye molecule comprises a detectable label.

TABLE 2
Summary of wavelengths used for photobleaching either through standard emission
filters in a microscope (2^{nd} column) or custom light sources (third column).
Excitation wavelengths are listed as a range for each fluorescent dye.

| | | Approximate | |
|---|---|---|---|
| | Wavelength Range | wavelength Range for | |
| | for Photobleaching | Photobleaching with | |
| | through standard | Custom Illumination | Wavelength Range - |
| Label/Dye Name | Excitation Filter (nm) | (nm) | Excitation Dye (nm) |
| Alexa Fluor 750 | 722-758 | 722-758 | 600-830 |
| Alexa Fluor 647 | 626-644 | 626-644 | 550-680 |
| Alexa Fluor 594 | 570-600 | 570-600 | 470-640 |
| Atto 565 | 554-568 | 554-568 | 450-600 |
| Alexa Fluor 555 | 535-575 | 535-575 | 500-580 |
| Alexa Fluor 532 / | 523-541 | 523-541 | 430-590 |
| Atto 532 | | | |
| Alexa Fluor 488 / | 454-496 | 454-496 | 400-550 |
| Atto 488 | | | |

The excitation wavelength can also be used to decolorize the dye; this happens if the dye is excited for a prolonged period of time—i.e. during photobleaching, a process which can be made more efficient with SPS, PDS and/or PMS.

A “range of light” or “light range” and like terminology refers to a range of wavelengths of light. In some embodiments, a light range comprises wavelengths that excite a detectable marker, and/or photobleach a detectable marker, such as a fluorophore. In embodiments, the specified light range is an “excitation range” and/or the specified light range is a “photobleaching range.” In some embodiments, an excitation range and photobleaching range are the same for a given detection molecule. In some embodiments, an excitation range and photobleaching range are overlapping for a given detection molecule. In some embodiments, an excitation range is within a photobleaching range for a given detection molecule. In some embodiments, a photobleaching range is within an excitation range for a given detection molecule. As used herein, a light range does not include white light. Exemplary light ranges are provided in Table 2.

White light can be effective with the photobleaching compositions and methods disclosed herein, and in some embodiments, white light is used, e.g., for non-selective photobleaching. However, for selective photobleaching applications, a particular light range, or set of light ranges is used.

In some embodiments, photobleaching a detection molecule according to the present disclosure inactivates the detection molecule; as used in this context, inactivation refers to the chemical alternation of an optical signal generator causing it to lose its ability to generate an optical signal.

As used herein, the term “radical initiator” refers to substances that possess weak bonds with small dissociation energies and therefore can produce radical species under mild conditions.

As used herein, the term “sulfate radical initiator” refers to a substance that can generate sulfate radicals (SO₄·⁻) upon dissociation under mild conditions and thus can promote radical reactions. The sulfate radical has a high oxidative potential and readily reacts with organic molecules (e.g., detection molecules). Examples of sulfate radical initiators are peroxymonosulfate and peroxydisulfate salts.

As used herein, the term “photosensitizer” refers to compounds that absorb light and transfer the energy from the incident light into another nearby molecule, either directly or by a chemical reaction.

An exemplary workflow is presented below, for multiplex imaging of proteins using antibody detection methods, and using the compositions and methods of the present disclosure to photobleach signal between rounds of target labeling and detection.

### Exemplary Workflow (see FIG. 3)

Sample Preparation and Antibody Staining: The sample is mounted on a glass carrier slide, dewaxed, and processed for antigen retrieval. Following multiple washes in appropriate buffers, the sample is then mounted in a fluidics chamber of an optical imaging system.

Autofluorescence signal detection: Background autofluorescence of the sample is imaged using the same channel settings as for fluorophore signal detection. Multiple background signals may be collected for multiple channels at this time.

Dilution of Fluorophore-Labeled Antibody: One or more fluorophore-labeled antibodies are diluted to the appropriate concentration using antibody diluent. Multiple proteins can be stained at the same time if the antibody is conjugated with spectrally distinct fluorophores.

Antibody Incubation: A working solution of the antibody is manually added to the sample and/or dispensed automatically by the imaging device. The sample is incubated for 1 hour at room temperature (RT) to allow binding of the antibody to its target.

Washing Steps: The sample is washed three times with phosphate-buffered saline (PBS). Each wash lasts 5 minutes to remove unbound antibodies.

Signal Detection: The antibody-fluorophore signal is imaged using a fluorescence microscope. The appropriate light source and filter sets are used for optimal signal detection. A single fluorophore or multiple different fluorophores can be imaged.

### Fluorescence Signal Removal

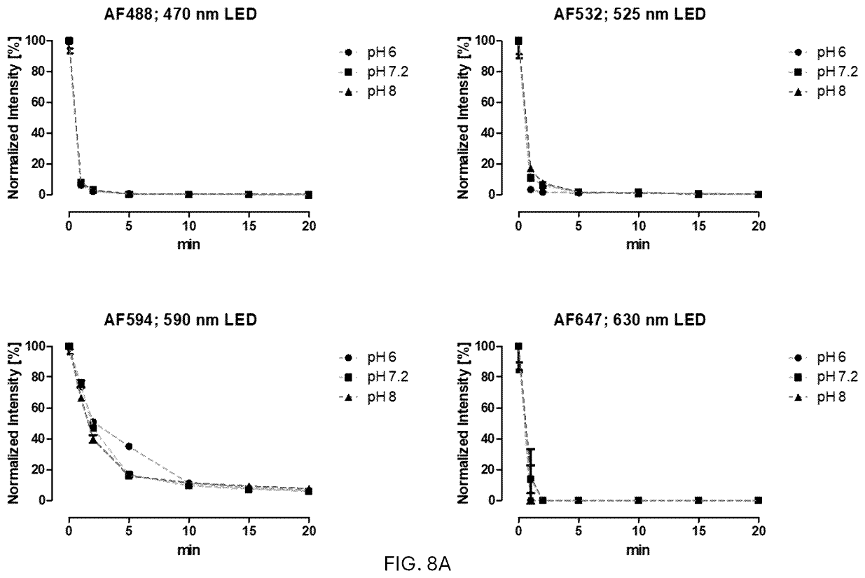

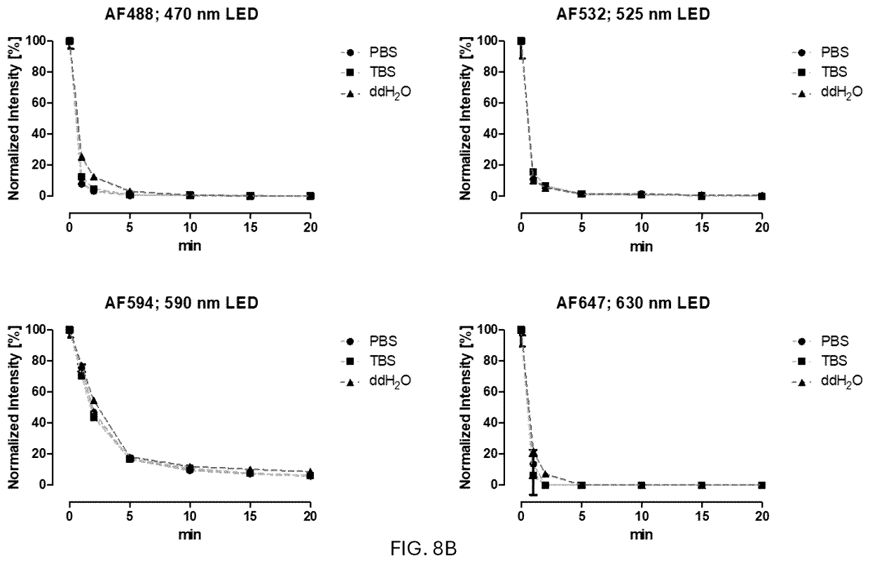

In one embodiment, sodium peroxydisulfate (and/or sodium peroxymonosulfate) treatment is as follows: 5 mg/ml sodium peroxydisulfate (SPS) in PBS is manually added or automatically dispensed onto the sample. The sample is then irradiated in one of the following ways: for 3-10 minutes under a light range of low to moderate intensity (irradiance 10-200 mW/cm²) that illuminates the whole sample chamber and is capable of exciting and irradiating all of the fluorophores selected (e.g. under white light, 420-750 nm). Alternatively, the sample may be irradiated for 5-10 seconds with the high-irradiance (600-1500 mW/cm²) light beam that originates from the microscope optics in the imaging system and is used to capture the sample image in a given field of view; irradiating the whole sample requires repeated repositioning of the sample over the beam of light. In some embodiments, prior to irradiation, the pH of the sample may be adjusted (if needed) to a pH ranging from about 5 to about 9 (see e.g., FIG. 8A). In some embodiments, the pH may be adjusted to a pH between about 6-9, 7-9, or between about 7-8 prior to irradiation. Methods and buffers for pH adjustment are well known in the art.

Washing: The sample is washed five times with PBS to completely remove SPS.

Background Fluorescence Imaging: An image of the background autofluorescence in the same channel that was previously used for irradiation is acquired to ensure complete signal removal.

Repeat for Subsequent Staining/Imaging Cycles: The entire process is repeated for each subsequent staining or imaging cycle.

### Exemplary, Non-Limiting Applications

Applications of the disclosed technology include, but are not limited to IF, mIF, FISH, smFISH (e.g. RNAscope™, HCR™ FISH, Stellaris™), Western Blotting, tyramide-based signal detection and amplification (e.g. Opal system, generic TSA), nanobody-based antibody detections (e.g. FlexAble, Nanotag). Furthermore, the disclosed technology enables combination of certain aforementioned applications into a single sequential workflow to enable the detection of multiple biomarker classes within a single tissue sample (e.g. sequential detection of protein epitopes with IF, and mRNA transcripts with smFISH methods).

### Exemplary, Non-Limiting Advantages

The disclosed technology offers several key advantages over other existing methods for removing fluorescence signals from a sample. Due to its reaction mechanism, PDS/PMS-enhanced photobleaching is selective and allows targeting specific reporter molecules in a sample based on their fluorophore excitation wavelength.

The reaction speed is determined by the irradiance of the target molecules with light in their specific excitation wavelength range. Molecules that do not undergo fluorescence excitation do not activate PDS/PMS and remain unchanged. Therefore, this method is comparably gentle and does not damage probes or target tissues.

Furthermore, enhanced photobleaching can inactivate fluorophores/dyes specifically designed for high photostability, which are typically resistant to conventional photobleaching procedures. It can decolorize any organic dye with fluorescent properties.

The compositions and methods disclosed herein also yield a reduction in inherent autofluorescence of biological samples (see e.g., FIG. 15).

Unlike H₂O₂ or LiBH₄-based chemical signal removal methods (IBEX, CyCIF), this reaction does not produce gas bubbles, allowing it to be used in enclosed microfluidic systems for reagent delivery onto samples.

These characteristics enable the technology to be used almost universally for removing fluorescence reporter signals across various multiplex imaging applications. It is the only method that can be easily integrated into automated cyclical workflows for optical, fluorescence-based biomarker imaging at the protein, transcriptome, and genome levels. The gentle and selective process also makes it suitable for use on sensitive sample materials like fresh-frozen tissues and fixed cell suspensions. The technology is fast and utilizes readily available, chemically stable reagents. The working solution is non-hazardous and can be stored for over a year at 4° C. This allows for the preparation, shipping, and prolonged storage of standardized, ready-to-use working solutions, increasing ease of use and reproducibility of experiments.

TABLE 3
Comparison between SPS/PMS enhanced photobleaching and other published
methods of chemical or photo-chemical signal removal.

| | Enhanced | | | | | | | | WO | |
|---|---|---|---|---|---|---|---|---|---|---|
| Method | Photobleaching | | CyCIF | | IBEX | | SPECTRE-Plex | | 2014/093455 | |
| Reagent | SPS/PMS | | H₂O₂ | | LiBH₄ | | m-CPBA | | Borate Salts | |
| Acute Toxicity¹ | Category 4 | | Category 4 | | Category 3 | | Category 4 | | Category 3 | |
| LD50 | 300-2000 mg/kg | | 300-2000 mg/kg | | 50-300 mg/kg | | 300-2000 mg/kg | | 50-300 mg/kg | |
| Effective | 5 mg/ml SPS | | 4.5% H₂O₂ | | 1 mg/ml LiBH₄ | | 1.72 mg/ml | | 10 | mM |
| concentration | | | 24 mM NaOH | | | | CPBA | | | |
| | | | | | | | 24 mM NaOH | | | |
| pH of working | 7 | | 12+ | | 10 | | 12+ | | 6-8 | |
| solution | | | | | | | | | | |
| Working | 12+ months at | | Immediate | | Immediate | | <36 | h | no info | |
| Solution | 4° C. (FIG. 19) | | use <1 h | | use <4 h | | | | | |
| stability | | | | | | | | | | |
| Exposure time | <1 | min | 30-60 | min | 15 | min | 3 | min | 20 | sec |

| Gas formation | No | | Yes (Oxygen) | | Yes (Hydrogen) | | No | | No |
|---|---|---|---|---|---|---|---|---|---|
| Compatibility | Yes | | No | | No | | Yes | | Yes |
| w/ | | | | | | | | | |
| microfluidics | | | | | | | | | |
| Target | Fluorophore | | No | | No | | No | | No |
| selectivity | excitation | | | | | | | | |
| Dye removal | All common | | No AF594, | | No AF594, | | No AF594, | | Only data for |
| effectiveness | organic | | AF568, AF546 | | AF568, AF546 | | AF568, AF546 | | Cy3, Cy5 and |
| | fluorophores | | | | | | | | Rhodamine |
| Sample | >50 | cycles | 2-20 | cycles | 20 | cycles | 40 | cycles | No data |

| | | | | | |
|---|---|---|---|---|---|
| integrity | | | | | |
| Compatibility | Yes | No data | Yes | No data | No data |
| w/ fresh frozen | | | | | |
| Reference | — | U.S. Pat. | Radtke et al.⁶ | Anderson et al.⁷ | WO |
| | | No. 7,741,046 | | | 2014/093455 |
| | | Lin et al.^{3, 16} | | | |

¹Acute Toxicity according to GHS classification for the pure compound.

### Definitions and Terminology

The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a probe” or “a label” should be interpreted to mean “one or more probes” and “one or more labels,” respectively, unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist of” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### Miscellaneous

The steps of the methods described herein can be performed in any suitable order unless otherwise indicated herein or otherwise clearly contradicted by context. The steps may be repeated or reiterated any number of times to achieve a desired goal unless otherwise indicated herein or otherwise clearly contradicted by context.

Preferred aspects of this invention are described herein. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Exemplary Embodiments

Disclosed herein are methods, kits, systems, platforms, and compositions for visualizing targets in biological samples. Several non-limiting embodiments of the present technology are provided below.
1. A method of probing multiple targets in a biological sample comprising:
(a) binding at least one target probe to one or more targets present in the biological sample that contains multiple targets;
(b) binding at least one reporter probe to at least one target probe bound in step (a);
(c) detecting a signal from at least one reporter probe bound in step (b);
(d) contacting the sample comprising the bound target probe of step (a) and the reporter probe of step (b) with a sulfate radical initiator that can engage in a photoreaction with a molecule capable of undergoing photoexcitation (i.e. the reporter probe of step (b))
wherein the said sulfate radical initiator, which in this embodiment consists of a peroxymonosulfate (PMS) or peroxydisulfate (PDS) salt, is represented by the following structural formula:

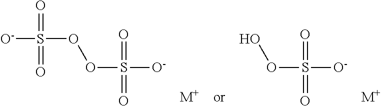

wherein M+ is selected from the group consisting of organic and inorganic cations;
(e) irradiating the reporter probe of step (b) leading to the production of sulfate radicals,
(f) repeating steps (a) through (e) above in an iterative manner until all targets in the biological sample in step (a) have been recorded.
2. The method of embodiment 1, wherein the target probe in step (a) comprises any molecule that is used to label epitopes or RNA transcripts within a biological sample. These include but are not limited to antibodies and RNA FISH probes.
3. The method of embodiment 1, wherein the reporter probe in step (b) comprises an optical signal generator, and the signal detected in step (c) is an optical signal.
4. The method of embodiment 1, wherein the probe in step (b) comprises a fluorescent signal generator, and the signal detected in step (c) is a fluorescent signal.
5. The method of embodiment 1, wherein irradiating the sample in step (e) is carried out in the presence of a buffer at pH of 5-9.
6. The method of embodiment 1, wherein irradiating the sample in step (e) is accomplished by exposing the sample to light which can excite the reporter probe, typically, a range of light encompassed by 350 nm to 1.3 mm in wavelength.
7. The method of embodiment 1, wherein irradiating the sample in step (e) is carried out in the presence of a buffer including a sulfate radical initiator, i.e. PMS and/or PDS.
8. A method of probing multiple targets in a biological sample comprising:
(a) binding multiple probes to multiple targets present in the biological sample including multiple targets, wherein the multiple probes include a first set of probes and a second set of probes;
(b) detecting a first set of signals from the first set of probes bound in step (a);
(c) contacting the sample comprising the bound probes of step (a-b) with a sulfate radical initiator, wherein said sulfate radical initiator is as defined in FIG. 3 of embodiment 1
(d) irradiating the sample of step (a)
(e) binding multiple probes from the second set to multiple targets present in the biological sample including multiple targets
(f) generating a second set of signals from the second set of probes bound in step (e)
(g) detecting the second set of signals.
9. The method according to embodiment 1, wherein the probe comprises a binder and a signal generator wherein the binder and the signal generator are embodied in a single entity.
10. The method according to embodiment 1, wherein the probe comprises a first binder, which binds to the target and a second binder, which binds to the first binder, and carries a signal generator.
11. The method according to embodiment 8, wherein the single entity is a small molecule probe.
12. An automated process for photoactivated chemical bleaching of a biological sample loaded and/or captured in a flow cell device, comprised of the following automated steps:
a) binding at least one probe to one or more targets present in the biological sample;
b) detecting a signal from the at least one reporter probe bound in step (a) or after;
c) filling the flow cell with a sulfate radical initiator, wherein said sulfate radical initiator is as defined in FIG. 3 of embodiment 1;
d) irradiating the sample by exposure to light to inactivate signals from the reporter probe;
e) optionally washing out the sulfate radical initiator; and repeating steps 11a) and 11b) with at least one other probe, for another round of imaging.

### EXAMPLES

The following Examples are illustrative and are not intended to limit the scope of the claimed subject matter.

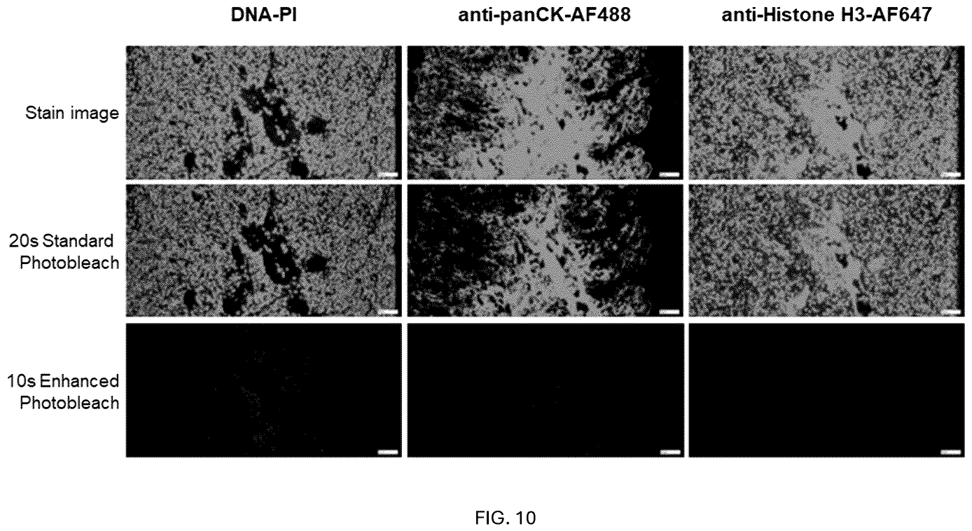

### Example 1—Methods Used to Produce the Data and Images in FIG. 10

Human formalin-fixed paraffin-embedded (FFPE) tonsil tissue was cut into 5 μm sections on a microtome. The sections were mounted on a microscopy slide, dried, and baked for 1 h at 60° C. The baked sections were stored at 4° C. One section was dewaxed by serial immersion in 2 changes of RotiClear (Carl Roth GmbH, Germany) for 5 minutes each, followed by rehydration in a graded alcohol series, 2×100% ethanol, 1×90%, 1×70%, 1×50% and finally in PBS for 5 minutes each. After rehydration, the sample was subjected to an antigen retrieval procedure in CC1 buffer (Roche, Germany) for 20 minutes at 95° C., followed by cooling in PBS at room temperature for 5 minutes. The tissue section was subsequently enclosed in a CellScape™ flow chamber which enabled rounds of on-instrument staining, imaging, and signal removal. The tissue was stained with fluorophore-labeled primary antibodies against pan-cytokeratin (clone AE-1/AE-3, AF488), Histone H3 (clone 1B1-B2, AF647) and propidium iodide (PI) DNA dye for 1 h at room temperature and imaged on the instrument; resultant data are shown in the top row of FIG. 10 and indicated as “Stain image”. The sample remained in PBS as was then photobleached for 20 sec through continuous exposure to white light passed through a 405 LP Edge Basic Longpass-Filter (420-750 nm, 800 mW/cm²). The stain was imaged again, and resultant data are shown in the middle row of FIG. 10 indicated as “20 s Standard Photobleach”. Finally, the flow chamber containing the sample was filled with PBS containing 5 mg/ml sodium peroxydisulfate and the photobleaching (800 mW/cm²) with white light was repeated for another 10 s, followed by imaging. The resultant data are shown in the bottom row of FIG. 10, indicated as “10 s Photobleach SPS” and they clearly demonstrate the efficacy of SPS mediated photobleaching in removing reporter signals from stained tissues.

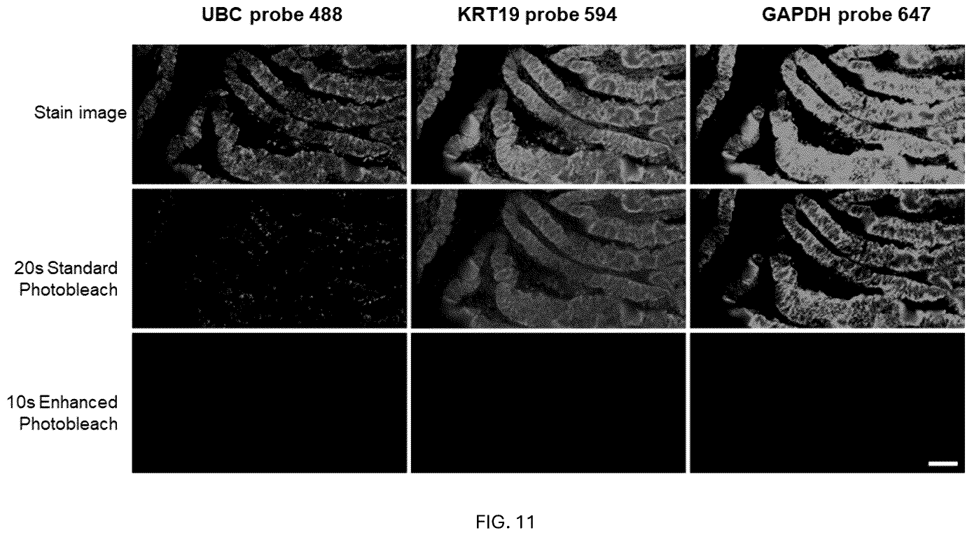

### Example 2—Methods Used to Produce the Data and Images in FIG. 11

For HCR™ Gold RNA-FISH, a FFPE tonsil sample was prepared as described above in Example 1. After antigen retrieval, the tissue sample was hybridized with HCR™ RNA-FISH probes against ubiquitin c (UBC), keratin 19 (KRT19), and Glyceraldehyde 3-phosphate dehydrogenase (GAPDH) transcripts for 2 h at 42° C. The tissue was then enclosed in a CellScape™ flow chamber. Detection of HCR™ Gold probes was achieved by labeling with target-specific fluorescently labeled HCR™ Gold amplifiers for signal generation for 2 h at 42° C.; samples were subsequently washed and imaged to produce the data in the top row of FIG. 11. After a 20 s photobleaching step through a 405 LP Edge Basic Longpass-Filter (420-750 nm, 800 mW/cm²) on the sample in PBS, the stain was imaged again; the resultant partial decolorization of the RNA detection signal is shown in the middle row of FIG. 11. Finally, the flow chamber was filled with PBS containing 5 mg/ml sodium peroxydisulfate and the photobleaching (800 mW/cm²) was repeated for 10 s, followed by imaging. The resulting complete decolorization of the RNA detection signal is shown in the bottom row of FIG. 11.

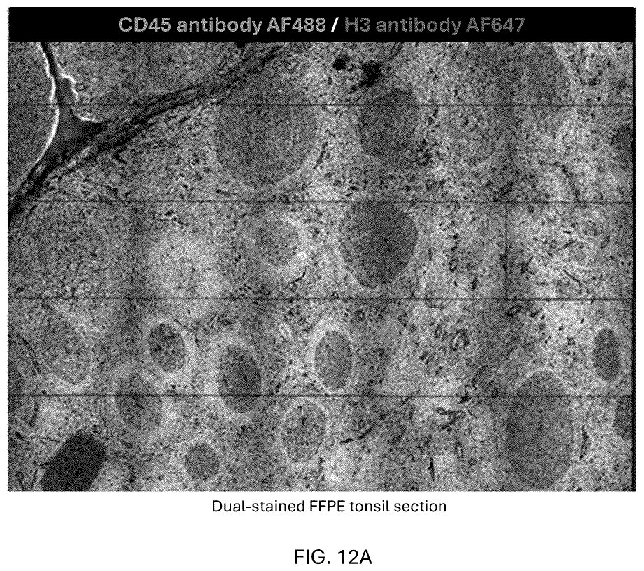

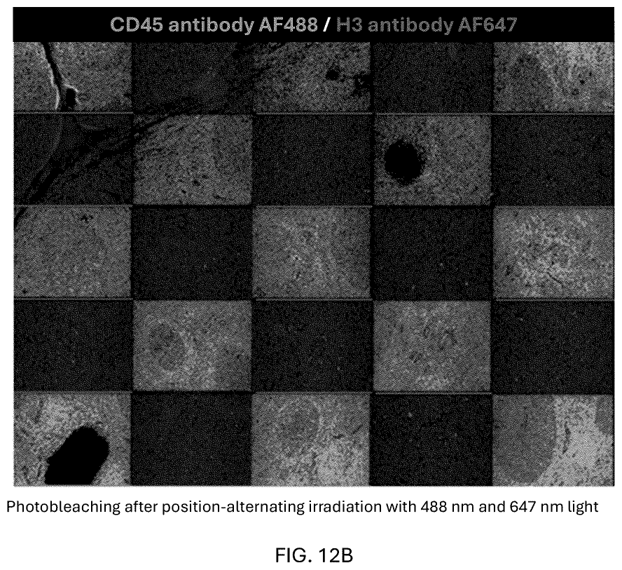

### Example 3—Methods Used to Produce the Data and Images in FIG. 12A-B

A FFPE tonsil sample was prepared as described above in Example 1. After antigen retrieval, the tissue sample was stained with fluorophore-labeled primary antibodies against CD45 (clone HI30, AF488) and Histone H3 (clone 1B1-B2, AF647) for 1 h at room temperature and then imaged to produce the data shown in FIG. 12A. Subsequently, the chamber was filled with 5 mg/ml sodium peroxydisulfate in PBS and every even numbered field-of-view was photobleached through the FS488 excitation filter (454-497 nm) and every odd numbered field-of-view was bleached through the FS647 excitation filter (626-644 nm). After bleaching, peroxydisulfate was washed out and all fields were imaged again to produce the checkerboard patterned image shown in FIG. 12B.

### Example 4—Methods Used to Produce the Data and Images in FIGS. 13 and 14

Serial normal and cancer tissue multi array sections (5 μm thickness) were prepared as described above in Example 1 up to the antibody staining step. Prior to antibody staining, the tissues underwent different treatments to assess possible effects of photobleaching on antibody labeling and staining performance. One section (control) was not photobleached before staining and followed the normal procedure outlined in Example 1 above. The second section (50× bleach cycles) was repeatedly photobleached (532-555 nm; 100 mW/cm² for 10 sec) with 5 mg/ml SPS in PBS for 50 times. All samples were then imaged to obtain background fluorescence pictures in each fluorescence channel from a similar region of interest for each serial section. DNA stains were performed for the 50× bleach cycles samples before the first photobleaching (cycle 1) and after the 50th photobleaching (cycle 50). Antibody and DNA staining was then performed in CellScape™ storage buffer for 1 h at room temperature with the following dilutions: (1) anti-CD3-AF488 [SP162] 1:500; (2) anti-panCK-AF532 [AE1/AE2] 1:500; (3) anti-FoxP3-AF594 [SP97] 1:100; (4) Anti-CD20-AF647 [L26] 1:500; (5) Sytox Orange DNA dye 1:106. Sections were then washed for 10 minutes with CellScape wash buffer under constant flow (0.5 ml/min) and imaging was performed with the same exposure and acquisition setting for each section. Antibody imaging data and the effect of SPS-mediated photobleaching on reporter signal generation are shown in FIG. 13. The effect of SPS mediated photobleaching on the tissue integrity is shown in FIG. 14.

### REFERENCES

1. Gao, Y. Catalyst-free activation of peroxides under visible LED light irradiation through photoexcitation pathway. J. Hazard. Mater. (2017).
2. Tsuneoka, Y., Atsumi, Y., Makanae, A., Yashiro, M. & Funato, H. Fluorescence quenching by high-power LEDs for highly sensitive fluorescence in situ hybridization. Front. Mol. Neurosci. 15, (2022).
3. Lin, J.-R., Fallahi-Sichani, M., Chen, J.-Y. & Sorger, P. K. Cyclic Immunofluorescence (CycIF), A Highly Multiplexed Method for Single-cell Imaging. Curr. Protoc. Chem. Biol. 8, 251-264 (2016).
4. Migliozzi, D. et al. Microfluidics-assisted multiplexed biomarker detection for in situ mapping of immune cells in tumor sections. Microsyst. Nanoeng. 5, 1-12 (2019).
5. Hsu, J., Nguyen, K. T., Bujnowska, M., Janes, K. A. & Fallahi-Sichani, M. Protocol for iterative indirect immunofluorescence imaging in cultured cells, tissue sections, and metaphase chromosome spreads. STAR Protoc. 5, 103190 (2024).
6. Radtke, A. J. et al. IBEX: an iterative immunolabeling and chemical bleaching method for high-content imaging of diverse tissues. Nat. Protoc. 17, 378-401 (2022).
7. Anderson, M. D. et al. SPECTREPlex: an automated, fast, high-resolution enabled approach for multiplexed cyclic imaging and tissue spatial analysis. Commun. Biol. 8, 636 (2025).
8. Black, S. et al. CODEX multiplexed tissue imaging with DNA-conjugated antibodies. Nat. Protoc. 16, 3802-3835 (2021).
9. Jhaveri, N. et al. Mapping the Spatial Proteome of Head and Neck Tumors: Key Immune Mediators and Metabolic Determinants in the Tumor Microenvironment. GEN Biotechnol. 2, 418-434 (2023).
10. Freeburne, E. et al. Spatial Single Cell Analysis of Proteins in 2D Human Gastruloids Using Iterative Immunofluorescence.
11. Jawahar Deen, A. and P. Making Multiplexed Imaging Flexible-Combining Essential Markers with Established Antibody Panels. Cell Rep. Methods.
12. Anipsitakis, G. P. & Dionysiou, D. D. Degradation of organic contaminants in water with sulfate radicals generated by the conjunction of peroxymonosulfate with cobalt. Environ. Sci. Technol. 37, 4790-4797 (2003).
13. Fang, G., Gao, J., Dionysiou, D. D., Liu, C. & Zhou, D. Activation of persulfate by quinones: free radical reactions and implication for the degradation of PCBs. Environ. Sci. Technol. 47, 4605-4611 (2013).
14. Matzek, L. W. Activated persulfate for organic chemical degradation: A review. (2016).
15. Lee, J., von Gunten, U. & Kim, J.-H. Persulfate-Based Advanced Oxidation: Critical Assessment of Opportunities and Roadblocks. Environ. Sci. Technol. 54, 3064-3081 (2020).
16. Lin, J. R. et al. Tissue Cyclic Immunofluorescence (t-CyCIF) version 3. https://www.protocols.io/view/tissue-cyclic-immunofluorescence-t-cycif-version-3-ch2ht8b6, (2025).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for detection of one or more targets in a biological sample, the method comprising:
a. contacting the biological sample with a detectable label and linking the detectable label to one or more targets;
b. detecting a signal from the detectable label;
c. after detection at step (b), contacting the sample of (b) with PMS, PDS, a salt thereof, or any combination thereof, wherein PMS and PDS are represented by the following formulas: 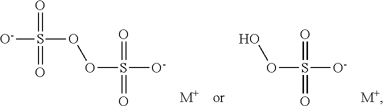
d. irradiating the sample of (c), wherein irradiating comprises exposing the sample to light.

2. The method of claim 1, wherein the light is a selected wavelength within the wavelength range required to detect the detectable label.

3. The method of claim 1, wherein the detectable label comprises a dye.

4. The method of claim 1, wherein step (a) comprises a target probe.

5. The method of claim 1, wherein the dye is linked to the target probe.

6. The method of claim 1, wherein step (a) further comprises a reporter probe, wherein the dye is linked to the reporter probe.

7. The method of claim 1, wherein the biological sample comprises a cell that expressed the dye.

8. The method of claim 1, wherein the target comprises RNA or DNA.

9. The method of claim 1, wherein the target comprises a protein.

10. The method of claim 1, wherein the target comprises a protein, and the target probe comprises an antibody.

11. The method of claim 1, wherein step (d) is carried out at a pH in the range of about 5 to about 9.

12. The method of claim 1, wherein step (c) comprises PMS or a salt thereof.

13. The method of claim 1, wherein step (c) comprises PDS or a salt thereof.

14. The method of claim 1, wherein the method does not comprise addition of an acid or an anti-oxidant buffer after step (b).

15. The method of claim 1, wherein the detectable label comprises a stain and wherein the stain comprises one or more of hematoxylin and eosin.

16. The method of claim 1, wherein irradiating is done with white light.

17. A method for sequential detection of multiple targets in a biological sample, the method comprising:
a. contacting a first target probe to the sample, wherein the first target probe is specific for a first target;
b. hybridizing or binding the first target probe to the first target in the sample;
c. detecting a first fluorescent detectable label, wherein the first fluorescent detectable label is linked to the first target probe;
d. after detection at step (c), contacting the sample of (c) with PMS, PDS, a salt thereof, or any combination thereof, wherein PMS and PDS are represented by the following formulas: 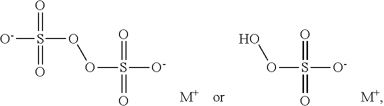
e. irradiating the sample of (d), wherein irradiating comprises exposing the sample to light;
f. repeating steps (a)-(e), comprising an Nth target probe and an Nth fluorescent detectable label different from the first probe and first fluorescent detectable label.

18. The method of claim 17, wherein the fluorescent detectable label is chemically linked to the target probe.

19. The method of claim 17, wherein step (c) comprises a reporter probe, wherein the fluorescent detectable label is linked to the reporter probe and wherein the reporter probe is configured to hybridize or bind to the target probe.

20. The method of claim 17, wherein step (e) is carried out at a pH in the range of about 5 to about 9.

21. The method of claim 17, wherein at step (e) a selected wavelength range of light is used, wherein the selected wavelength range is within the wavelength range required to detect the first fluorescent detectable label; and wherein at step (f) a selected wavelength range of light is used, wherein the selected wavelength range is within the wavelength range required to detect the Nth fluorescent detectable label.

22. The method of claim 17, wherein irradiating at step (e), (f), or both, is done with white light.

23. A method for detecting a plurality of targets in a biological sample, the method comprising:
a. contacting a plurality of target probes to the sample comprising N subsets of target probes, wherein each probe of the subsets of target probes is specific for different target;
b. hybridizing or binding the target probes to the plurality of targets in the sample;
c. detecting a first fluorescent detectable label, wherein the first fluorescent detectable label is linked to a first subset of the plurality of target probes;
d. after detection at step (c), contacting the sample of (c) with PMS, PDS, a salt thereof, or any combination thereof, wherein PMS and PDS are represented by the following formulas: 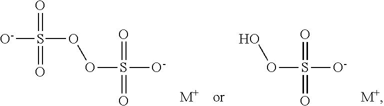
e. irradiating the sample of (d), wherein irradiating comprises exposing the sample to a light, optionally wherein the light is a selected wavelength range of light, wherein the selected wavelength range is within the wavelength range required to detect the first fluorescent detectable label;
f. repeating steps (c)-(e), comprising an Nth fluorescent detectable label linked to an Nth probe, wherein the Nth fluorescent detectable label and the Nth subset of target probes are different from the first subset of target probe and the first fluorescent detectable label.

24. The method of claim 23, wherein the fluorescent detectable labels are chemically linked to the target probes.

25. The method of claim 23, wherein the fluorescent detectable labels are components of detection molecules that are different from the target probes, and are configured to hybridize or bind to the target probes.

26. The method of claim 23, wherein step (d) comprises PMS or a salt thereof.

27. The method of claim 23, wherein step (d) comprises PDS or a salt thereof.

28. The method of claim 23, wherein the method does not comprise addition of an acid or an anti-oxidant buffer after step (c).

29. The method of claim 23, wherein at step (e) a selected wavelength range of light is used, wherein the selected wavelength range is within the wavelength range required to detect the first fluorescent detectable label; and wherein at step (f) a selected wavelength range of light is used, wherein the selected wavelength range is within the wavelength range required to detect the Nth fluorescent detectable label.

30. The method of claim 23, wherein irradiating at step (e), (f), or both, is done with white light.
